(19) United States
(12) Patent Application Publication
(10) Pub. No.: US 2016/0044901 A1
(43) Pub. Date: Feb. 18, 2016

(54) Transgenic Animals With Customizable Traits

(71) Applicant: AgGenetics, Inc, Nashville, TN (US)

(72) Inventor: James WEST, Nashville, TN (US)

(21) Appl. No.: 14/777,108

(22) PCT Filed: Mar. 15, 2014
(86) PCT No.: PCT/US2014/029918

§ 371 Date: Sep. 15, 2015

Related U.S. Application Data
(63) Continuation-in-part of application No. 13/837,405, filed on Mar. 15, 2013.
Application No. 13/837,405 is a continuation-in-part of application No. 13/768,760, filed on Feb. 15, 2013.
(60) Provisional application No. 61/598,987, filed on Feb. 15, 2012.

(51) Int. Cl. A01K 67/027; C12N 15/85

(52) U.S. Cl. CPC A01K 67/0275; C12N 15/8509; A01K 2227/101; A01K 2207/05; A01K 2217/206

(57) ABSTRACT

Disclosed are materials and methods for creating customizable traits in animals. In the demonstration of the principle of the subject invention, a keratin-14 specific promoter is used with red fluorescent protein in the loxp cassette, dominant black (ΔG23) beta defensin 103 in the pigment cassette, and an SV40 (with intron) polyadenylation sequence. When Cre recombinase (or HTNCre) is applied to the animal's skin in a carrier base (e.g., lipid bilayers), fur is permanently genetically modified to turn back in the shape in which the HTNCre was applied.

### CROSS-REFERENCE TO RELATED APPLICATION

This application is a continuation-in-part application of U.S. application Ser. No. 13/837,405, filed Mar. 15, 2013, which is a continuation-in-part application of U.S. application Ser. No. 13/768,760, filed Feb. 15, 2013, which claims the priority benefit of U.S. Application Ser. No. 61/598,987, filed Feb. 15, 2012, all of which are incorporated herein by reference in their entirety.

The Sequence Listing for this application is labeled SeqList-14Mar13_ST25.txt which was created on Mar. 14, 2013 and is 85 KB. The entire content of the sequence listing is incorporated herein by reference in its entirety.

### FIELD OF THE INVENTION

This invention relates to genetic modification of animals in order to effect controlled changes in specified traits such as, for example, changes in skin and/or fur pigmentation.

### BACKGROUND OF THE INVENTION

The only pigment synthesized in most animal species, and the only pigment at all in mammalian species, is melanin. There are two classes of melanin: pheomelanin, which produces a blond or red color, and eumelanin, which produces a dark brown or black color. Both classes of melanin are synthesized from tyrosine, but their synthetic pathways diverge after production of dopaquinone.

The primary switch controlling whether a particular melanocyte produces pheomelanin or eumelanin is the melanocortin receptor (MC1R). MC1R polymorphisms also appear to be the primary determinant of red or blond pheomelanin.

The melanocortin receptor can be activated by any of the melanocyte-stimulating hormones (MSH), most commonly by α-MSH, but also by β-defensin 103. Conversely, activation of MC1R can be inhibited through expression of the agouti signaling protein (ASP). The β-defensin 103 signal is dominant over the ASP signal and the ASP signal is dominant over the MSH signal. Further, there are mutations and polymorphisms in all of these genes that increase or decrease their activity. Coat color is further modulated by multiple modulators of melanin production and transport, including tyrosinase (TYR), the tyrosinase transporter OCA2, and the ubiquitination gene HERC2, among others.

Plants make multiple additional classes of pigments, including chlorophyll, carotenoids, anthrocyanins, and betalains. Of these pigments, carotenoids can be most easily transferred to animals because production of carotenoid dyes in plants relies on a precursor that can also be found in animals. Geranylgeranyl pyrophosphate is an intermediate in the HMG-CoA reductase pathway, and is used as a precursor for synthesizing steroids and sterols. With the addition of 4-5 plant proteins, geranylgeranyl pyrophosphate can instead be used as a precursor for synthesizing carotene yellow and orange, or torulene red. The transfer of plant pigments to animals has occurred in nature: the aphid A. pisum has several genes somehow transferred from fungi. Carotenoids have also been produced in genetically-engineered yeast, which belong to the animal kingdom.

The only modification of surface pigment ever attempted by genetic intervention in multicellular animals is the wholesale change of animals from light to dark or dark to light. No more detailed patterns have been created. Carotene dyes have never been naturally found in animals more complicated than aphids, and have never been engineered into any multicellular animal. Before the present invention, customizable color or patterns in the skin or fur of animal species have not been created.

### BRIEF SUMMARY

The subject invention provides materials and methods for creating customizable traits in animals. For example, the subject invention provides materials and methods for creating customizable patterns in the skin and/or fur of animals. In other embodiments, the customizable trait can involve the length and/or texture of animal skin or fur. Alternatively, the subject invention can be used to effect controlled changes in the texture, structural strength, and/or length of animal nail, claw, and/or horn.

In one specific embodiment, the methods of the subject invention comprise introducing into the cells of an animal a genetic construct comprising a keratin-14 specific promoter, red fluorescent protein in a loxp cassette, dominant black (ΔG23) beta defensin 103 in a pigment cassette, and an SV40 (with intron) polyadenylation sequence. When a composition comprising Cre recombinase (or HTNCre) is then applied to the skin of an animal having the genetic construct, the fur of the animal will turn black where the composition was applied. In this way, the fur is permanently genetically modified to turn color in a desired shape.

Thus, in one embodiment the subject invention provides a method of creating customizable permanent patterns in the skin and/or fur of animals.

In another embodiment the subject invention provides methods for producing multicolor patterns in the skin and/or fur of animal species.

In a further embodiment the subject invention provides a method of creating customizable patterns in the skin and/or fur of animal species such that the animal would continue to grow fur to sustain those colors throughout its lifetime.

The invention also provides methods of creating customizable predefined patterns of stripes that are heritable within that animal.

In one embodiment, the present invention provides transdermal application of one or more activating factors to drive recombination and permanent transgene expression in the transgenic animals of the present invention. In certain specific embodiments, the activating factors useful according to the present invention include, but are not limited to, recombinase proteins, small molecules (such as, doxycycline, cumate, ecdysone, etc) capable of inducing the expression of recombinase, viruses that capable of inducing the expression of recombinase, and nucleic acid (such as DNA) constructs that drive the expression of recombinase.

In certain embodiments, the activating factor is applied to the surface of the animal skin, either alone or in a carrier solution (e.g., liposomes, solvents, mixtures containing DMSO, etc.). In one embodiment, the activating factor is applied intradermally (such as with the use of a tattoo needle) or subdermally.

In one embodiment, the transgenic animal comprises one or more exogenous nucleic acid molecules including, but not limited to, pigmentation-related genes, coat/hair quality genes (such as genes for controlling the length and/or curliness of animal hair), genes related to nail/claw or horn quality (such as a nucleic acid molecule encoding cross-linking keratin), and genes for synthesis and/or expression of plant pigments in animal cells.

In certain embodiments, promoters useful according to the present invention include, but are not limited to, skin-specific promoters (e.g., keratin specific promoter), melanocyte specific promoters (e.g., MCR promoter), constitutive promoters (e.g., beta-globin promoter, CMV promoter), and promoters responsive to circulating factors such as NF-kB, interferon gamma, estrogen, and glucocorticoids.

In certain embodiments, the present invention provides the use of multiple types of recombinase targets to allow specific activation of different genes selectively through application of different recombinases. In one embodiment, multiple recombinase targets are used to allow multiple colors to be created after birth of the animal.

In one embodiment, in a transgenic dog with a naturally golden fur, Cre recombinase activates the production of dog fur with black color, and Flp recombinase activates the production of dog fur with red color.

In one embodiment, the present invention provides the use of native promoters to drive coat pigmentation without the need for an external activating factor. In a specific embodiment, the native promoter relates to defining somite boundaries in animal development.

In one embodiment, the native heterologous promoter is used to create coat patterns in a different species, such as, for example, using the Tabby or Ticked promoters found in cats to drive coat coloration in dogs.

In certain embodiments, the present invention provides genetically modified animals with coat patterns that are permanently customizable after birth.

In certain embodiments, the present invention provides genetically modified animals with coat colors not normally found in mammals.

In certain embodiments, the present invention provides genetically modified cattle, sheep or other animals that have permanent identification marks (such as, a number or a bar code) growing in their coat.

In certain embodiments, the present invention provides genetically modified cattle, sheep or other animals that have “invisible” marks in their coat that can change color in response to changes in health or physiological conditions.

In one embodiment, the transgenic cattle or sheep or other animals can express one symbol on the coat or fur, wherein that symbol can change color if the animal has chronic activation of NF-kB, and another symbol that can change color if the animal has chronic activation of interferon-gamma.

In certain embodiments, the present invention provides genetically modified animals born with coat colors and patterns not normally found in their native species.

In one embodiment, the present invention provides a transgenic bovine animal of the black Angus breed with white or near-white coat or fur.

### BRIEF DESCRIPTION OF THE FIGURES

FIG. 1 is a schematic depiction of an embodiment of a genetic construct for effecting color change in animals.

FIG. 2 is a schematic depiction of an embodiment of a genetic construct for creating customizable patterns and color in animal skin or fur.

FIG. 3 is a schematic depiction of an embodiment of genetic constructs for creating customizable patterns and color in animal skin or fur.

FIG. 4 is a schematic depiction of one embodiment of a genetic construct for creating customizable patterns and color in the skin or fur of mice. The construct comprises a Keratin14 promoter, which is expressed in all skin fibroblasts, to drive the dominant black form of signaling molecule β-Def103. The expression of β-Def103 is blocked by a LoxP excisable nucleic acid encoding ring finger protein (RFP), which is used as a marker. The nucleic acid molecule encoding RFP is not required for the construct, and can be replaced by STOPs.

FIG. 5 (A) shows that beta-Def103 expression activated by transdermal application of recombinase creates a genetically permanent alteration in the coat or fur of an adult mouse. (B) Fine control of marking alteration can be achieved: a mouse with a single narrow black line is created by injection of recombinase. The change in skin/fur pattern has persisted through multiple cycles of coat regrowth, indicating successful alteration of resident stem cells.

FIG. 6 shows that coat color of the transgenic mice can be titrated through the amount of recombinase applied to the mouse skin. Increasing the amounts of recombinase applied to the mice increases the level of transgene expression and eumelanin activation.

FIG. 7 is a schematic depiction of one embodiment of a genetic construct for knock-in cattle with spermatozoa-specific expression of enhanced green fluorescent protein (EGFP), and recombinase-mediated melanocyte-specific expression of a dominant negative Rab7. A nucleic acid sequence encoding neomycin resistance biomarker protein, which can be excised through PIGGYBAC™ transposons, is placed in the center of the construct, and the construct is flanked by short homology arms.

FIG. 8 shows a depiction of a Black Angus heifer genetically engineered to express a customizable identification pattern in the skin.

### BRIEF DESCRIPTION OF THE SEQUENCES

SEQ ID NO:1 is the amino acid sequence of a bifunctional enzyme CarRP-like isoform 1 [Acyrthosiphon pisum] (GenBank Accession No. XP_{—}001943170).

SEQ ID NO:2 is the amino acid sequence of a bifunctional enzyme CarRP-like [Acyrthosiphon pisum] (GenBank Accession No. XP_{—}001950787).

SEQ ID NO:3 is the amino acid sequence of a lycopene cyclase/phytoene synthase-like [Acyrthosiphon pisum] (GenBank Accession No. XP_{—}001950868).

SEQ ID NO:4 is the amino acid sequence of a phytoene dehydrogenase-like [Acyrthosiphon pisum] (GenBank Accession No. XP_{—}001943225).

SEQ ID NO:5 is the amino acid sequence of a phytoene dehydrogenase-like [Acyrthosiphon pisum] (GenBank Accession No. XP_{—}001950764).

SEQ ID NO:6 is the amino acid sequence of a phytoene dehydrogenase-like [Acyrthosiphon pisum] (Genbank Accession No. XP001946689).

SEQ ID NO:7 is the amino acid sequence of a phytoene dehydrogenase-like [Acyrthosiphon pisum] (Genbank Accession No. XP_{—}001943938).

SEQ ID NO:8 is the amino acid sequence of a melanocortin 1 receptor (GenBank Accession No. EDL11741).

SEQ ID NO:9 is the amino acid sequence of an alpha melanocyte stimulating hormone (MSH).

SEQ ID NO:10 is the amino acid sequence of a beta melanocyte stimulating hormone (MSH).

SEQ ID NO:11 is the amino acid sequence of a beta melanocyte stimulating hormone (MSH).

SEQ ID NO:12 is the amino acid sequence of a gamma melanocyte stimulating hormone (MSH).

SEQ ID NO:13 is the amino acid sequence of a β-defensin protein (GenBank Accession No. AAT67592).

SEQ ID NO:14 is the amino acid sequence of an agouti signaling protein precursor (GenBank Accession No. NP_{—}056585).

SEQ ID NO:15 is the amino acid sequence of a tyrosinase (TYR) (GenBank Accession No. BAA00341).

SEQ ID NO:16 is the amino acid sequence of a melanocyte-specific transporter protein (GenBank Accession No. Q62052).

SEQ ID NO:17 is the amino acid sequence of a rab protein geranylgeranyltransferase component A2 (GenBank Accession No. NP_{—}067325).

SEQ ID NO:18 is the amino acid sequence of a ras-related protein Rab-7a (GenBank Accession No. NP_{—}033031).

SEQ ID NO:19 is the amino acid sequence of a probable E3 ubiquitin-protein ligase (HERC2) (GenBank Accession No. NP_{—}084390).

SEQ ID NO:20 is the amino acid sequence of a Pmel17 protein [Gallus gallus] (GenBank Accession No. AAT58250).

SEQ ID NO:21 is the amino acid sequence of a Pmel17 protein [Gallus gallus] (GenBank Accession No. AAT58246).

SEQ ID NO:22 is the amino acid sequence of a Pmel17 protein [Gallus gallus] (GenBank Accession No. AAT58249).

### DETAILED DISCLOSURE

The subject invention provides materials and methods for creating customizable traits in animals. For example, the subject invention provides materials and methods for creating customizable patterns in the skin and/or fur of animals. In other embodiments, the customizable trait can involve the length and/or texture of animal skin or fur. Alternatively, the subject invention can be used to effect controlled changes in the texture, structural strength, and/or length of animal hair, nail, claw and/or horn.

In one specific embodiment, the methods of the subject invention comprise introducing into the cells of an animal a genetic construct comprising a keratin-14 specific promoter, red fluorescent protein in a loxp cassette, dominant black (ΔG23) beta defensin 103 in a pigment cassette, and an SV40 (with intron) polyadenylation sequence. When a composition comprising Cre recombinase (or HTNCre) is then applied to the skin of an animal having the genetic construct, the fur of the animal turns black where the composition is applied. In this way, the fur is permanently genetically modified to turn color in a desired shape.

Thus, in one embodiment the subject invention provides a method of creating customizable permanent patterns in the skin and/or fur of animals.

In another embodiment the subject invention provides methods for producing multicolor patterns in the skin and/or fur of animal species.

In a further embodiment the subject invention provides a method of creating customizable patterns in the skin and/or fur of animal species such that the animal would continue to grow fur to sustain those colors throughout its lifetime.

The invention also provides methods of creating customizable predefined patterns of stripes that are heritable within that animal.

In one embodiment, the transgenic animal has heritable soft claws or soft hair or fur.

In another embodiment, the present invention provides cells, tissues, or parts (such as skin, hair, fur) of the transgenic animal, and uses thereof. In one embodiment, the transgenic animal has customizable color and/or patterns in the skin and/or fur, and the skin or fur can be subsequently removed from the transgenic animal for production of hides, fur, leather, etc, useful for production of clothing, rugs, shoes, horse tack, horse harness, upholstery, and other leather goods.

Transgenic Animals with Customizable Traits

In one embodiment, the present invention provides a transgenic animal with customizable traits, wherein the transgenic animal (such as in its genome) comprises:

an exogenous nucleic acid molecule encoding a protein of interest, wherein the nucleic acid molecule is operably linked to a promoter and is under the control of an inducible gene expression system that requires the presence of an inducing agent to activate gene expression;
wherein the expression of the exogenous nucleic acid molecule is inhibited in the absence of the inducing agent.

In certain embodiments, the exogenous nucleic acid molecule that encodes a protein is selected from pigment proteins; proteins involved in the synthesis and/or transport of pigments; luminescent (such as fluorescent) proteins; proteins involving the length and/or texture of animal skin or fur; and proteins involved in the texture, structural strength, and/or length of animal nail, claw, and/or horn.

In one specific embodiment, the present invention provides a transgenic animal with customizable fur or skin pigmentation, wherein the genome of the transgenic animal comprises:

an exogenous nucleic acid molecule encoding a pigment protein of interest or a protein involved in the synthesis and/or transport of a pigment of interest, wherein the exogenous nucleic acid molecule is operably linked to a promoter and is under the control of an inducible gene expression system that is a site-specific recombination system,

wherein the site-specific recombination system inhibits the expression of the first nucleic acid molecule in the absence of site-specific recombination.

The expression of the exogenous nucleic acid molecule can be induced after the application of, for example, a recombinase to the transgenic animal.

In one embodiment, the exogenous nucleic acid molecule, the promoter, and/or the site-specific recombination system are contained in a pigmentation construct.

In one embodiment, the genome of the transgenic animal comprises an exogenous nucleic acid molecule whose expression is under the control of a Cre/LoxP recombination system, wherein the Cre/LoxP recombination system prevents the expression of the exogenous nucleic acid molecule. In one specific embodiment, the Cre/LoxP recombination system comprises a lox-stop-lox (LSL) sequence.

In one embodiment, the pigmentation construct is transferred into cells, such as fertilized ova. The pigmentation construct can be transferred into fertilized ova using any conventional means, including, but not limited to, lentivirii, pronuclear injections, and intracytoplasmic sperm injection (ICSI).

In certain embodiments, one or more pigmentation constructs are introduced into the genome of the transgenic animal. The pigmentation construct can comprise more than one exogenous nucleic acid molecule, each nucleic acid molecule encoding a protein of interest.

In certain embodiments, the genome of the transgenic animal comprises more than one inducible gene expression systems to control the expression of the nucleic acid molecules of interest.

In one specific embodiment, the present invention provides a transgenic animal with customizable traits (such as fur or skin pigmentation), wherein the genome of the transgenic animal comprises:

a first exogenous nucleic acid molecule encoding a protein of interest (such as a pigmentation protein) operably linked to a first promoter and under the control of a loxP site, wherein the loxP site prevents the expression of the first exogenous nucleic acid molecule in the absence of Cre recombinase protein; and

a second nucleic acid molecule encoding a Cre recombinase protein, operably linked to a second promoter.

In another specific embodiment, the present invention provides a transgenic animal with customizable traits (such as fur or skin pigmentation), wherein the genome of the transgenic animal comprises:

a first exogenous nucleic acid molecule encoding a protein of interest (such as pigmentation protein) operably linked to a first promoter and under the control of a loxP site, wherein the loxP site prevents the expression of the first nucleic acid molecule in the absence of Cre recombinase protein;

a second nucleic acid encoding a reverse tRA (rtTA), operably linked to a second promoter; and

a third nucleic acid molecule encoding a Cre recombinase protein, operably linked to a third promoter under the control of a TetO operator.

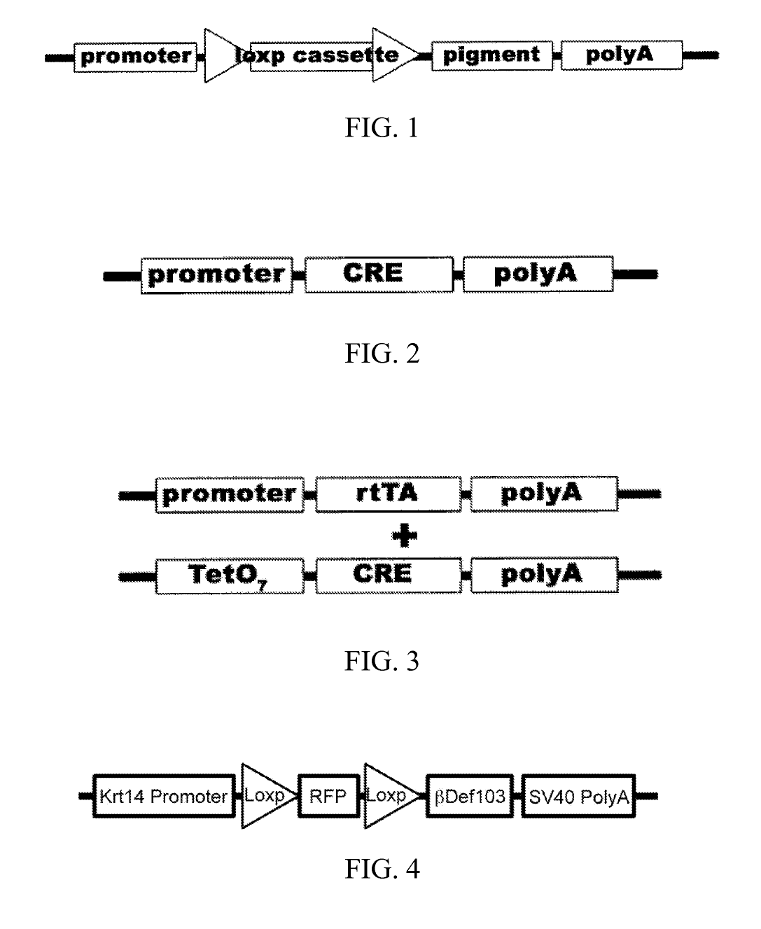

FIGS. 2 and 3 show an embodiment of the expression constructs, wherein the expression of the exogenous nucleic acid molecule of interest (such as pigment proteins and proteins involved in the synthesis of biological pigments) is under the control of the Cre-LoxP recombination system and a tetracycline (Tet)-controlled transcription activation system.

In one specific embodiment, doxycycline (or ecdysone, etc) mixed with DMSO carrier is applied to a transgenic animal with a gold fur color, whereby the color of the transgenic animal turns red; subsequently, Cre or HTNCre mixed with DMSO is applied to the transgenic animal to produce a black color.

In another specific embodiment, the genome of the transgenic animal comprises an exogenous nucleic acid molecule the expression of which is under the control of a tetracycline (Tet)-controlled transcriptional activation system.

In another embodiment, the present invention provides a transgenic animal with customizable traits (such as fur or skin pigmentation), wherein the genome of the transgenic animal comprises: a first exogenous nucleic acid molecule encoding a protein of interest (such as a pigmentation protein) operably linked to a first promoter and under the control of an inducible gene expression system (e.g., a tetracycline (Tet)-controlled transcriptional activation system), wherein the inducible gene expression system, in its inactivated state (absent of induction), prevents the expression of the first nucleic acid molecule.

In one embodiment, the present invention provides a transgenic animal with customizable traits, wherein the transgenic animal (such as in its genome) comprises: a dominantly acting, exogenous nucleic acid molecule encoding a protein of interest, wherein the nucleic acid molecule is operably linked to a promoter.

In one embodiment, the present invention provides a transgenic animal having a coat or fur color that is different from a wild-type animal of the same species, wherein the transgenic animal (such as in its genome) comprises: a dominantly acting, exogenous nucleic acid molecule encoding a protein selected from pigment proteins; proteins involved in the synthesis and/or transport of pigments; and luminescent (such as fluorescent) proteins.

In one embodiment, the present invention provides a transgenic animal with coat or fur having a color of interest, wherein the transgenic animal comprises:

a dominantly acting, exogenous nucleic acid molecule operably linked to a promoter, wherein the exogenous nucleic acid encodes a protein selected from proteins involved in melanosome assembly, proteins involved in the synthesis of melanin, proteins involved in the transport of melanin, proteins involved in melanocyte development and/or migration, and proteins involved in the synthesis and/or transport of biological pigments; and/or an exogenous inhibitory RNA coding sequence of interest, operably linked to a promoter, wherein the exogenous inhibitory RNA coding sequence of interest interferes with the expression of a dominantly acting wild-type nucleic acid molecule of the animal, wherein the wild-type nucleic acid molecule encodes a protein selected from proteins involved in melanosome assembly, proteins involved in the synthesis of melanin, proteins involved in the transport of melanin, proteins involved in melanocyte development and/or migration, and proteins involved in the synthesis and/or transport of biological pigments.

In one embodiment, the exogenous nucleic acid molecule is not present in the cells of the wild-type animal of the same species. In one embodiment, while the exogenous nucleic acid molecule is present in the cells of the wild-type animal of the same species, it is present at a different location in the wild-type genome. In one embodiment, while the exogenous nucleic acid molecule is present in the cells of the wild-type animal of the same species, it is expressed in different cell types in the wild-type animal. In another embodiment, while the exogenous nucleic acid molecule is present in the cells of the wild-type animal of the same species, the transgenic animal has additional copies of the exogenous nucleic acid, when compared to the wild-type animal of the same species.

The transgenic animal can be of any species, including, but not limited to, mammalian species including, but not limited to, domesticated and laboratory animals such as dogs, cats, mice, rats, guinea pigs, and hamsters; livestock such as horses, cattle, pigs, sheep, goats, ducks, geese, and chickens; primates such as apes, chimpanzees, orangutans, humans, and monkeys; fish; amphibians such as frogs and salamanders; reptiles such as snakes and lizards; and other animals such as fox, weasels, rabbits, mink, beavers, ermines, otters, sable, seals, coyotes, chinchillas, deer, muskrats, and possum. In certain embodiments, the animal is not a human.

Creation of Transgenic Bovine Animals with “Non-Black” Coat or Fur

In one embodiment, the present invention provides a transgenic bovine animal with white or near-white coat or fur, wherein the transgenic animal (such as in its genome) comprises:

a dominantly acting, exogenous nucleic acid molecule, operably linked to a promoter, wherein the dominantly acting, exogenous nucleic acid encodes a protein selected from proteins that inhibit melanosome assembly, proteins that inhibit the synthesis of melanin, proteins that inhibit the transport of melanin, and proteins that inhibit melanocyte development and/or migration; and/or

an exogenous inhibitory RNA coding sequence of interest, operably linked to a promoter, wherein the exogenous inhibitory RNA coding sequence encodes an inhibitory RNA that interferes with the expression of a dominantly acting wild-type nucleic acid molecule of the animal, wherein the wild-type nucleic acid molecule encodes a protein selected from proteins involved in melanosome assembly, proteins involved in the synthesis of melanin, proteins involved in the transport of melanin, and proteins involved in melanocyte development and/or migration.

In a preferred embodiment, the bovine animal is of the black Angus breed. In certain embodiments, transgenic bovine animals of the present invention can include, but are not limited to, domesticated cattle, bison, and buffalos (e.g., water buffalo, African buffalo).

In one embodiment, the dominantly acting, exogenous nucleic acid molecule is a dominantly acting white allele.

The term “allele,” as used herein, refers to its ordinary meaning that is an alternative form of a gene, usually arising from mutations, that is located at a specific position on a specific chromosome.

Dominant white is a group of genetically related coat or color phenotypes that are present in various breeds of wild-type animals, such as for example, chicken, horses, mice, dogs, and cats.

In one embodiment, the dominant white allele is a nucleic acid molecule encoding a Pmel17. Pmel17 (also called GP100 and Silv) is a melanocyte/melanoma-specific glycoprotein that plays a critical role in melanosome development. In certain embodiments, Pmel17 proteins useful according to the present invention can be from, for example, chicken, horses, mice, dogs, and cats. In one specific embodiment, the dominant white allele encodes a Pmel17 protein of white chicken.

In one embodiment, the dominant white allele is a nucleic acid molecule encoding Rab7.

In certain embodiments, the exogenous inhibitory RNA interferes with the expression of melanocortin receptor (MC1R), melanocyte stimulating hormones (MSH) (e.g., α-MSH, β-MSH, γ-MSH), β-defensin 103, agouti signaling protein (ASP), tyrosinase (TYR), melanocyte-specific transporter protein, Ras-related protein Rab-7, rab protein geranylgeranyltransferase component A2, and probable E3 ubiquitin-protein ligase (HERC2).

In one embodiment, the transgenic bovine animal further comprises an inducible gene expression system. In one specific embodiment, the expression of the exogenous nucleic acid molecule or the expression of the exogenous inhibitory RNA coding sequence is under the control of the inducible system. In one embodiment, the inducible gene expression system is a site-specific recombination system (e.g., the Lox/P system).

In a preferred embodiment, the transgenic bovine animal has white or near-white coat color. In certain embodiments, the transgenic bovine animal has non-black colors, such as, gray, pink, brown, and yellow.

### Pigment Proteins

The term “pigment protein,” as used herein, refers to a protein comprising a pigment. The term “pigment,” as used herein, refers to a material that does not emit light but changes the color of reflected or transmitted light as the result of wavelength-selective absorption; this physical process differs from fluorescence, phosphorescence, and other forms of luminescence, in which a material emits light. Pigment proteins include, but are not limited to, chromoproteins such as cytochromes and flavoproteins.

### Luminescent Proteins

The term “luminescent protein,” as used herein, refers to a protein that emits light. Luminescent proteins useful according to the present invention include, but are not limited to, fluorescent proteins including, but not limited to, green fluorescent protein, yellow fluorescent protein, cyan fluorescent protein, and red fluorescent protein; and phosphorescent proteins. Fluorescent proteins are members of a class of proteins that share the unique property of being self-sufficient to form a visible wavelength chromophore from a sequence of three amino acids within their own polypeptide sequence. A variety of luminescent proteins, including fluorescent proteins, are publicly known. Fluorescent proteins useful according to the present invention include, but are not limited to, the fluorescent proteins disclosed in U.S. Pat. No. 7,160,698, U.S. Application Publication Nos. 2009/0221799, 2009/0092960, 2007/0204355, 2007/0122851, 2006/0183133, 2005/0048609, 2012/0238726, 2012/0034643, 2011/0269945, 2011/0223636, 2011/0152502, 2011/0126305, 2011/0099646, 2010/0286370, 2010/0233726, 2010/0184116, 2010/0087006, 2010/0035287, 2007/0021598, 2005/0244921, 2005/0221338, 2004/0146972, and 2001/0003650, all of which are hereby incorporated by reference in their entireties.

### Proteins Involved in the Synthesis of Biological Pigments

Proteins involved in the synthesis and/or transport of biological pigments include, but are not limited to, the wild-type or mutant forms of melanocortin receptor (MC1R), melanocyte stimulating hormones (MSH) (e.g., α-MSH, β-MSH, γ-MSH), β-defensin 103, agouti signaling protein (ASP), tyrosinase (TYR), melanocyte-specific transporter protein, Ras-related protein Rab-7, rab protein geranylgeranyltransferase component A2, probable E3 ubiquitin-protein ligase (HERC2), Pmel17, and melanophilin (MLPH).

In certain embodiments, the genome of the transgenic animal comprises an exogenous nucleic acid molecule encoding a protein involved in the synthesis of a biological pigment.

Nucleic acid molecules encoding proteins involved in the synthesis and/or transport of biological pigments can be derived from genes including, but not limited to, the dominant MC1R E92K and the agouti gene.

In certain embodiments, the genome of the transgenic animal comprises a nucleic acid molecule encoding a protein involved in the synthesis and/or transport of biological pigments including, but not limited to, melanins (e.g., pheomelanin, eumelanin); urochrome; chlorophyll; bilirubin; biliverdin; phycobilin; phycoerythrobilin; stercobilin; urobilin; hemocyanin; hemoglobin; myoglobin; luciferins; carotenoids, including hematochromes, carotenes (e.g., alpha and beta carotene, lycopene, rhodopsin), xanthophylls (e.g., canthaxanthin, zeaxanthin, lutein); phytochrome; phycobiliproteins (e.g., R-phycoerythrin (R-PE), B-phycoerythrin (B-PE), C-phycocyanin (CPC), allophycocyanin (APC)); polyene enolates; and flavonoids.

Carotenoid pigments in yellow, red, or orange can be synthesized in animals that express phytoenesynthases, desaturases, and cyclases, as described in Moran (2010) and Verdoes (2003). In one embodiment, the carotenoid dyes are synthesized using geranylgeranyl pyrophosphate as a substrate.

Proteins involved in the synthesis and/or transport of biological pigments include, but are not limited to, bifunctional enzyme CarRP-like isoform 1 [Acyrthosiphon pisum] (such as, GenBank Accession No. XP_{—}001943170 (SEQ ID NO:1)), bifunctional enzyme CarRP-like [Acyrthosiphon pisum] (such as, GenBank Accession No. XP_{—}001950787 (SEQ ID NO:2)), lycopene cyclase/phytoene synthase-like [Acyrthosiphon pisum] (such as GenBank Accession No. XP_{—}001950868 (SEQ ID NO:3)), phytoene dehydrogenase-like [Acyrthosiphon pisum] (such as, GenBank Accession No. XP_{—}001943225 (SEQ ID NO:4)), phytoene dehydrogenase-like [Acyrthosiphon pisum] (such as, GenBank Accession No. XP_{—}001950764 (SEQ ID NO:5)), phytoene dehydrogenase-like [Acyrthosiphon pisum] (such as, GenBank Accession No. XP001946689 (SEQ ID NO:6)), and phytoene dehydrogenase-like [Acyrthosiphon pisum] (such as, GenBank Accession No. XP_{—}001943938 (SEQ ID NO:7)).

Proteins involved in the synthesis and/or transport of biological pigments can be of any animal origin (such as mouse, porcine, human, horse, dog, cat, mouse, chicken) including, but not limited to, melanocortin 1 receptor (such as, GenBank Accession No. EDL11741 (SEQ ID NO:8)), alpha melanocyte stimulating hormones (MSH) (such as, SEQ ID NO:9), beta melanocyte stimulating hormones (MSH) (such as, SEQ ID NO:10, SEQ ID NO:11), gamma melanocyte stimulating hormones (MSH) (such as, SEQ ID NO:12), β-defensin (such as, GenBank Accession No. AAT67592 (SEQ ID NO:13)), agouti signaling protein precursor (such as, GenBank Accession No. NP_{—}056585 (SEQ ID NO:14)), tyrosinase (TYR) (such as, GenBank Accession No. BAA00341 (SEQ ID NO:15)), melanocyte-specific transporter protein (such as, GenBank Accession No. Q62052 (SEQ ID NO:16)), rab proteins geranylgeranyltransferase component A2 (such as, GenBank Accession No. NP_{—}067325 (SEQ ID NO:17)), ras-related protein Rab-7a (such as, GenBank Accession No. NP_{—}033031 (SEQ ID NO:18)), and probable E3 ubiquitin-protein ligase (HERC2) (such as GenBank Accession No. NP_{—}084390 (SEQ ID NO:19)), and melanophilin (MLPH) (such as GenBank Accession No. NP_{—}44379848.2).

In certain embodiments, proteins involved in the synthesis and/or transport of biological pigments can be proteins having at least 80% identity, or having any percent identity higher than 80% (such as at least 85%, 87%, 90%, 92%, 93%, 94%, 95%, 96%, 97%, 98%, or 99%), to any of SEQ ID NOs: 1-22.

Various Pmel17 proteins sequences are publicly available, such as via the GenBank database. Pmel17 proteins useful according to the present invention include, but are not limited to, Gallus gallus Pmel17 having amino acid sequences, such as SEQ ID NOs: 20-22.

Proteins Involved in the Texture, Structural Strength, and/or Length of Hair, Nail, Claw and/or Horn

In certain embodiment, the transgenic animal expresses a nucleic acid molecule encoding a protein that alters hair quality (such as straight or curly hair) or length. In one embodiment, conditional over-expression of WNT3 or DVL2 in the outer root sheath induces shorter hair in animals. In certain embodiments, the transgenic animal expresses a nucleic acid molecule encoding a protein involves that the texture, structural strength, and/or length of animal hair, nail, claw and/or horn.

Proteins involved in controlling the texture, structural strength, and/or length of animal hair, nail, claw and/or horn include keratin proteins, including, but not limited to, keratin 1, keratin 2, keratin 2A, keratin HB6, keratin 3, keratin 4, keratin 5, keratin 6, keratin 7, keratin 8, keratin 9, keratin 10, keratin 11, keratin 12, keratin 13, keratin 14, keratin 15, keratin 16, keratin 17, keratin 18, keratin 19, keratin 20, keratin 23, keratin 24, keratin 25, keratin 26, keratin 27, keratin 28, keratin 31, keratin 32, keratin 33, keratin 34, keratin 35, keratin 36, keratin 37, keratin 38, keratin 39, keratin 40, keratin 71, keratin 72, keratin 73, keratin 74, keratin 75, keratin 76, keratin 77, keratin 78, keratin 79, keratin 80, keratin 81, keratin 82, keratin 83, keratin 84, keratin 85, and keratin 86.

Modification of Amino Acid and/or Polynucleotide Sequences

The amino acid sequences of a variety of pigment proteins; proteins involved in the synthesis and/or transport of pigments; proteins involving the length and/or texture of animal skin or fur; and proteins involved in the texture, structural strength, and/or length of animal nail, claw, and/or horn, are publically available, such as via the GenBank database. The present invention encompasses the use of such proteins.

Polynucleotides and polypeptides within the scope of the subject invention can also be defined in terms of identity with those sequences that are specifically exemplified herein. The sequence identity will typically be greater than 60%, preferably greater than 75%, more preferably greater than 80%, even more preferably greater than 90%, and can be greater than 95%. The identity of a sequence can be 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% as compared to a sequence exemplified herein.

Unless otherwise specified, as used herein percent sequence identity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, wordlength=12, to obtain sequences with the desired percent sequence identity. To obtain gapped alignments for comparison purposes, Gapped BLAST can be used as described in Altschul et al. (1997). When utilizing BLAST and Gapped BLAST programs, the default parameters of the respective programs (NBLAST and XBLAST) can be used. See NCBI/NIH website.

### Promoter Elements

In certain embodiments in accordance with the present invention, the nucleic acid molecule is operably linked to a constitutive, inducible, or tissue-specific promoter.

The term “constitutive promoter,” as used herein, refers to its ordinary meaning that is an unregulated promoter that allows for continual transcription of its associated gene. Constitutive promoters useful according to the present invention include, but are not limited to, cytomegalovirus (CMV) promoter, CMV-chicken beta actin promoter, ubiquitin promoter, JeT promoter, SV40 promoter, beta globin promoter, elongation Factor 1 alpha (EF1-alpha) promoter, RSV promoter, and Mo-MLV-LTR promoter.

Promoters useful according to the present invention include, but are not limited to, universal promoters (e.g., Rosa26); tissue-specific promoters, such as keratinocyte specific promoters (e.g., Keratin 14); melanocyte specific promoters (e.g., promoter of the melanocortin 1 receptor (MCR1) gene); and dermal papilla-specific promoters. Promoters useful according to the present invention include melanocyte specific promoters and matrix-cell specific promoters.

Keratinocyte specific promoters include, but are not limited to, promoters of keratin 1, keratin 2, keratin 2A, keratin HB6, keratin 3, keratin 4, keratin 5, keratin 6, keratin 7, keratin 8, keratin 9, keratin 10, keratin 11, keratin 12, keratin 13, keratin 14, keratin 15, keratin 16, keratin 17, keratin 18, keratin 19, keratin 20, keratin 23, keratin 24, keratin 25, keratin 26, keratin 27, keratin 28, keratin 31, keratin 32, keratin 33, keratin 34, keratin 35, keratin 36, keratin 37, keratin 38, keratin 39, keratin 40, keratin 71, keratin 72, keratin 73, keratin 74, keratin 75, keratin 76, keratin 77, keratin 78, keratin 79, keratin 80, keratin 81, keratin 82, keratin 83, keratin 84, keratin 85, and keratin 86 gene.

In certain embodiments, promoters useful according to the present invention include, but are not limited to, promoters inducing gene expression in the presence of an endogenous biological factor of interest, such as NF-KB, interferon-gamma, estrogen, and or glucocorticoids.

Promoters useful according to the present invention include, but are not limited to, promoters inducing gene expression in the presence of an infectious agent of interest, such as a virus, bacteria, and/or protozoa.

In one embodiment, a dermal papilla-specific promoter is used for creating customizable pigmentation, color, or pattern in cells derived from somites; useful promoters include, but are not limited to, a Ripply2 promoter, a Tabby promoter, and a Ticked promoter. The choice of promoters can be determined by performing multiple species comparisons and/or using the extent of well-conserved promoter elements. In certain embodiments, the promoter element further comprises a nucleic acid molecule encoding a reporter protein, which is expressed in response to the administration of drugs, and/or a metabolic state or circulating levels of biomarkers in the transgenic animal. In one embodiment, the promoter induces expression of a protein of interest (such as a pigment protein and/or a protein involved in the synthesis of a biological pigment) in response to the presence of a physiological state of interest in the transgenic animal, such as for example, cardiac stress, increased levels of circulating cytokines, and/or increased steroid presence or activity.

### Inducible Expression Systems

The inducible gene expression systems useful according the present invention include, but are not limited to, site-specific recombination systems including, but not limited to, a Cre-LoxP recombination system, a FLP-FRT recombination system; a tetracycline (Tet)-controlled transcription activation system; an ecdysone inducible system; a heat shock on/off system; a lacO/IPTG system; a cumate repressor protein CymR system; a nitroreductase system; coumermycin/novobiocin-regulated system; a RheoSwitch Ligand RSL1 system; a chimeric bipartite nuclear receptor expression system; a GAL4 system; sterol or steroid or synthetic steroid inducing/repressing system; and any combination thereof.

In one embodiment, the inducible system useful according to the present invention is a Cre-LoxP recombination system. The genome of the transgenic animal can comprise an exogenous nucleic acid molecule whose expression is under the control of a Cre/LoxP recombination system, wherein the Cre/LoxP recombination system prevents the expression of the exogenous nucleic acid molecule. In one specific embodiment, the Cre/LoxP recombination system comprises a lox-stop-lox (LSL) sequence.

The Cre-LoxP recombination system is a site-specific recombination technology useful for performing site-specific deletions, insertions, translocations, and inversions in the DNA of cells or transgenic animals. The Cre recombinase protein (encoded by the locus originally named as “causes recombination”) consists of four subunits and two domains: a larger carboxyl (C-terminal) domain and a smaller amino (N-terminal) domain. The loxP (locus of X-over P1) is a site on the Bacteriophage P1 and consists of 34 bp. The results of Cre-recombinase-mediated recombination depend on the location and orientation of the loxP sites, which can be located cis or trans. In case of cis-localization, the orientation of the loxP sites can be the same or opposite. In case of trans-localization, the DNA strands involved can be linear or circular. The results of Cre recombinase-mediated recombination can be excision (when the loxP sites are in the same orientation) or inversion (when the loxP sites are in the opposite orientation) of an intervening sequence in case of cis loxP sites, or insertion of one DNA into another or translocation between two molecules (chromosomes) in case of trans loxP sites. The Cre-LoxP recombination system is known in the art, see, for example, Andras Nagy, Cre recombinase: the universal reagent for genome tailoring, Genesis 26:99-109 (2000).

The Lox-Stop-Lox (LSL) cassette prevents expression of the transgene in the absence of Cre-mediated recombination. In the presence of Cre recombinase, the LoxP sites recombine, and the stop cassette is deleted. The Lox-Stop-Lox (LSL) cassette is known in the art. See, Allen Institute for Brain Science, Mouse Brain Connectivity Altas, Technical White Paper: Transgenic Characterization Overview (2012).

In certain embodiments, the loxP site further comprises a reporter gene encoding gene (e.g., lacz, GFP) and/or a nucleic acid molecule encoding a second pigmentation protein (e.g., if the first pigmentation contains a dominantly active MC1R, another agouti gene could be flanked by the loxP site).

Tetracycline (Tet)-controlled transcriptional activation is a method of inducible expression where transcription is reversibly controlled by the presence or absence of the antibiotic tetracycline or one of its derivatives (e.g., doxycycline). Gene expression is activated as a result of binding of the Tet-off or Tet-on protein to tetracycline response elements (TREs) located within an inducible promoter. Both the Tet-on and Tet-off proteins activate gene expression. The Tet-Off protein activates gene expression in the absence of a tetracycline derivative—doxycycline (Dox), whereas the Tet-on protein activates gene expression in the presence of Dox.

In the Tet-off system, the tetracycline transactivator (tTA) protein, which is created by fusing the TetR (tetracycline repressor) protein (obtainable from Escherichia coli bacteria) with the VP16 protein (obtainable from the Herpes Simplex Virus), binds on DNA at a TetO operator. Once bound the TetO operator activates the promoter coupled to the TetO operator, thereby activating the transcription of the nearby gene. Tetracycline derivatives bind tTA and render it incapable of binding to TRE sequences, thereby preventing transactivation of target genes.

In the Tet-On system, when the tTA protein is bound by doxycycline, the doxycycline-bound tTA is capable of binding the TetO operator. Thus, the introduction of doxycyline to the system initiates the transcription of the genetic product. The Tet-on system is sometimes preferred for the faster responsiveness.

The reverse tTA (rtTA) is a complementary genetic module for rapid gene activation by addition of Dox (Tet-on). See Kistner et al., Doxycycline-mediated quantitative and tissue-specific control of gene expression in transgenic mice, Proc. Natl. Acad. Sci. U.S.A. Vol. 93, pp. 10933-10938 (1996).

The Tet-on advanced transactivator (also known as rtTA2^{S}-M2) is an alternative version of Tet-On that shows reduced basal expression, and functions at a 10-fold lower Dox concentration than Tet-on. In addition, its expression is considered to be more stable in eukaryotic cells due to being human codon optimized and utilizing three minimal transcriptional activation domains. Tet-on 3G (also known as rtTA-V10) is similar to Tet-on Advanced, and is human codon optimized and composed of three minimal VP16 activation domains. The Tet-on 3G is sensitive to 100-fold less Dox than the original Tet-on.

In one embodiment of a tetracycline-responsive regulatory expression element, a tetracycline-controlled reverse transactivator (rtTA) comprises a tetR (e.g., from Escherichia coli Tn10); a mammalian transcription factor VP 16 transactivating domain serving as an effector; and a tissue-specific promoter controlling the rtTA effector transcription. In the presence of doxycycline, the rtTA binds to a (TeTO)₇ operator (a seven tandemly repeated TetO sequence) placed upstream of a CMV promoter that drives expression of a transgene. As a result, transgene expression can be switched on or off by administration and withdrawal of doxycycline.

### Expression Constructs

The present invention also provides expression constructs, vectors, as well as host cells useful for producing transgenic animals.

As used herein, the term “expression construct” refers to a combination of nucleic acid sequences that provides for transcription of an operably linked nucleic acid sequence. Expression constructs of the invention also generally include regulatory elements that are functional in the intended host cell in which the expression construct is to be expressed. Thus, a person of ordinary skill in the art can select regulatory elements for use in, for example, bacterial host cells, yeast host cells, plant host cells, insect host cells, mammalian host cells, and human host cells. Regulatory elements include promoters, transcription termination sequences, translation termination sequences, enhancers, and polyadenylation elements.

In certain embodiments, the present invention provides expression constructs for customizing color and/or pattern of animals, including pigmentation constructs and patterning constructs.

FIG. 1 shows an embodiment of a pigmentation construct comprising a promoter, a loxP cassette, a nucleic acid molecule encoding a pigment protein of interest or a protein involved in the synthesis and/or transport of a pigment of interest, and polyadenylation sequence. In one embodiment of the pigmentation construct, the expression of the pigment protein of interest or a protein involved in the synthesis and/or transport of a pigment of interest is activated by application of Cre recombinase.

In another embodiment, the present invention provides a pattern construct. FIG. 2 shows an embodiment of a patterning construct. In one embodiment, the promoter of the patterning construct is derived from a gene specific to somite boundary specification. In one embodiment, the promoter is selected from a Ripply2 promoter, a Tabby promoter, and a Ticked promoter. In one embodiment, the transgenic animal comprises a pigmentation construct and a patterning construct. In one embodiment, the transgenic animal has customizable constitutive vertical stripes on the dorsal dermis.

In one embodiment, the genome of the transgenic animal comprises:

1) pigmentation construct as shown in FIG. 1, wherein the promoter is a melanocyte-specific promoter and the nucleic acid molecule encodes a protein involved in the synthesis of a red pigment (such as ASIP or MC1R);

2) a pigmentation construct as shown in FIG. 1, wherein the promoter is a dermal papilla-specific promoter and the nucleic acid molecule encodes—defensin 103; and

3) a set of constructs as shown in FIG. 3, wherein the promoter is a melanocyte-specific promoter.

An expression construct of the invention can comprise a promoter sequence operably linked to a polynucleotide sequence encoding a peptide of the invention. Promoters can be incorporated into a polynucleotide using standard techniques known in the art. Multiple copies of promoters or multiple promoters can be used in an expression construct of the invention. In a preferred embodiment, a promoter can be positioned about the same distance from the transcription start site as it is from the transcription start site in its natural genetic environment. Some variation in this distance is permitted without substantial decrease in promoter activity. A transcription start site is typically included in the expression construct.

As used herein, the term “operably linked” refers to a juxtaposition of the components described wherein the components are in a relationship that permits them to function in their intended manner. In general, operably linked components are in contiguous relation. Sequence(s) operably-linked to a coding sequence may be capable of effecting the replication, transcription and/or translation of the coding sequence. For example, a coding sequence is operably-linked to a promoter when the promoter is capable of directing transcription of that coding sequence.

A “coding sequence” or “coding region” is a polynucleotide sequence that is transcribed into mRNA and/or translated into a polypeptide. For example, a coding sequence may encode a polypeptide of interest. The boundaries of the coding sequence are determined by a translation start codon at the 5′-terminus and a translation stop codon at the 3′-terminus.

The term “promoter,” as used herein, refers to a DNA sequence operably linked to a nucleic acid sequence to be transcribed such as a nucleic acid sequence encoding a desired molecule. A promoter is generally positioned upstream of a nucleic acid sequence to be transcribed and provides a site for specific binding by RNA polymerase and other transcription factors. In specific embodiments, a promoter is generally positioned upstream of the nucleic acid sequence transcribed to produce the desired molecule, and provides a site for specific binding by RNA polymerase and other transcription factors.

In addition to a promoter, one or more enhancer sequences may be included such as, but not limited to, cytomegalovirus (CMV) early enhancer element and an SV40 enhancer element. Additional included sequences are an intron sequence such as the beta globin intron or a generic intron, a transcription termination sequence, and an mRNA polyadenylation (pA) sequence such as, but not limited to, SV40-pA, beta-globin-pA, the human growth hormone (hGH) pA and SCF-pA.

In one embodiment, the expression construct comprises polyadenylation sequences, such as polyadenylation sequences derived from bovine growth hormone (BGH) and SV40.

The term “polyA” or “p(A)” or “pA” refers to nucleic acid sequences that signal for transcription termination and mRNA polyadenylation. The polyA sequence is characterized by the hexanucleotide motif AAUAAA. Commonly used polyadenylation signals are the SV40 pA, the human growth hormone (hGH) pA, the beta-actin pA, and beta-globin pA. The sequences can range in length from 32 to 450 bp. Multiple pA signals may be used.

In one embodiment, the genetic construct comprises a nucleic acid molecule encoding a selection marker, such as neomycin resistance biomarker protein, which can be excised through PIGGYBAC™ transposons. In one embodiment, the construct is flanked by short homology arms.

The term “vector” is used to refer to any molecule (e.g., nucleic acid, plasmid, or virus) used to transfer coding information (e.g., a polynucleotide of the invention) to a host cell.

The terms “expression vector” and “transcription vector” are used interchangeably to refer to a vector that is suitable for use in a host cell (e.g., a subject's cell) and contains nucleic acid sequences that direct and/or control the expression of exogenous nucleic acid sequences. Expression includes, but is not limited to, processes such as transcription, translation, and RNA splicing, if introns are present. Vectors useful according to the present invention include plasmids, viruses, BACs, YACs, and the like. Particular viral vectors illustratively include those derived from adenovirus, adeno-associated virus and lentivirus.

As used herein, the term “isolated” molecule (e.g., isolated nucleic acid molecule) refers to molecules which are substantially free of other cellular material, or culture medium when produced by recombinant techniques, or substantially free of chemical precursors or other chemicals when chemically synthesized.

The term “recombinant” is used to indicate a nucleic acid construct in which two or more nucleic acids are linked and which are not found linked in nature.

The term “nucleic acid” as used herein refers to RNA or DNA molecules having more than one nucleotide in any form including single-stranded, double-stranded, oligonucleotide or polynucleotide.

The term “nucleotide sequence” is used to refer to the ordering of nucleotides in an oligonucleotide or polynucleotide in a single-stranded form of nucleic acid.

The term “expressed” refers to transcription of a nucleic acid sequence to produce a corresponding mRNA and/or translation of the mRNA to produce the corresponding protein. Expression constructs can be generated recombinantly or synthetically or by DNA synthesis using well-known methodology.

The term “regulatory element” as used herein refers to a nucleotide sequence which controls some aspect of the expression of an operably linked nucleic acid sequence. Exemplary regulatory elements illustratively include an enhancer, an internal ribosome entry site (IRES), an intron, an origin of replication, a polyadenylation signal (pA), a promoter, a transcription termination sequence, and an upstream regulatory domain, which contribute to the replication, transcription, post-transcriptional processing of a nucleic acid sequence. Those of ordinary skill in the art are capable of selecting and using these and other regulatory elements in an expression construct with no more than routine experimentation.

In one embodiment, the construct of the present invention comprises an internal ribosome entry site (IRES). In one embodiment, the expression construct comprises kozak consensus sequences.

Optionally, a reporter gene is included in the transgene construct. The term “reporter gene” as used herein refers to a gene that is easily detectable when expressed, for example, via chemiluminescence, fluorescence, colorimetric reactions, antibody binding, inducible markers, ligand binding assays, and the like. Exemplary reporter genes include but are not limited to green fluorescent protein. The production of recombinant nucleic acids, vectors, transformed host cells, proteins and protein fragments by genetic engineering is well known.

If desired, the vector may optionally contain flanking nucleic sequences that direct site-specific homologous recombination. The use of flanking DNA sequences to permit homologous recombination into a desired genetic locus is known in the art. At present it is preferred that up to several kilobases or more of flanking DNA corresponding to the chromosomal insertion site be present in the vector on both sides of the encoding sequence (or any other sequence of this invention to be inserted into a chromosomal location by homologous recombination) to assure precise replacement of chromosomal sequences with the exogenous DNA. See e.g. Deng et al, 1993, Mol. Cell. Biol 13(4):2134-40; Deng et al, 1992, Mol Cell Biol 12(8):3365-71; and Thomas et al, 1992, Mol Cell Biol 12(7):2919-23. It should also be noted that the cell of this invention may contain multiple copies of the gene of interest.

Transformed host cells are cells which have been transformed or transfected with vectors containing nucleic acid constructs of the invention and may or may not transcribe or translate the operatively associated nucleic acid of interest.

### RNA Interference Cassette for Customization of Animal Traits

In another embodiment, the present invention provides a transgenic animal with customizable traits, wherein the genome of the transgenic animal comprises:

an exogenous inhibitory RNA coding sequence of interest, operably linked to a promoter and under the control of an inducible gene expression system that requires the presence of an inducing agent to activate gene expression;

wherein the expression of the exogenous inhibitory RNA coding sequence of interest is inhibited in the absence of the inducing agent.

In certain embodiments, the exogenous inhibitory RNA coding sequence of interest interferes with the expression of a nucleic acid sequence encoding pigment proteins; proteins involved in the synthesis and/or transport of pigments; proteins involving the length and/or texture of animal skin or fur; luminescent (such as fluorescent) proteins; and proteins involved in the texture, structural strength, and/or length of animal nail, claw, or horn texture.

In one embodiment, an exogenous inhibitory RNA coding sequence encodes an siRNA that interferes with the expression of cross-linking actin) in the nails, thereby producing genetically-engineered animals (such as cats) with nails that are soft instead of sharp.

In one embodiment, the RNAi construct comprises an siRNA that interferes with the expression of a nucleic acid molecule encoding cross-linking keratin), operably linked to a promoter specific to the cross-linking keratin, and is under the control of a reporter gene flanked by loxP sites.

Keratin proteins involved in the texture, structural strength, and/or length of animal hair, nail, claw and/or horn include, but are not limited to, keratin 1, keratin 2, keratin 2A, keratin HB6, keratin 3, keratin 4, keratin 5, keratin 6, keratin 7, keratin 8, keratin 9, keratin 10, keratin 11, keratin 12, keratin 13, keratin 14, keratin 15, keratin 16, keratin 17, keratin 18, keratin 19, keratin 20, keratin 23, keratin 24, keratin 25, keratin 26, keratin 27, keratin 28, keratin 31, keratin 32, keratin 33, keratin 34, keratin 35, keratin 36, keratin 37, keratin 38, keratin 39, keratin 40, keratin 71, keratin 72, keratin 73, keratin 74, keratin 75, keratin 76, keratin 77, keratin 78, keratin 79, keratin 80, keratin 81, keratin 82, keratin 83, keratin 84, keratin 85, and keratin 86.

In one embodiment, the present invention provides a microRNA cassette comprising the siRNA coding sequence and a 3′ UTR sequence.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA which direct the degradative mechanism to other similar RNA sequences. Alternatively, RNAi can be initiated by the hand of man, for example, to silence the expression of endogenous target genes, such as PKC-ι.

As used herein, the term “small interfering RNA” (“siRNA”) (also referred to in the art as “short interfering RNAs”) refers to an RNA (or RNA analog) comprising between about 10-50 nucleotides (or nucleotide analogs) which is capable of directing or mediating RNA interference.

As used herein, a siRNA having a “sequence sufficiently complementary to a target mRNA sequence to direct target-specific RNA interference (RNAi)” means that the siRNA has a sequence sufficient to trigger the destruction of the target mRNA (e.g., PKC-ι mRNA) by the RNAi machinery or process. “mRNA” or “messenger RNA” or “transcript” is single-stranded RNA that specifies the amino acid sequence of one or more polypeptides. This information is translated during protein synthesis when ribosomes bind to the mRNA.

The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester linkage between 5′ and 3′ carbon atoms. The terms “nucleic acid” or “nucleic acid sequence” encompass an oligonucleotide, nucleotide, polynucleotide, or a fragment of any of these, DNA or RNA of genomic or synthetic origin, which may be single-stranded or double-stranded and may represent a sense or antisense strand, peptide nucleic acid (PNA), or any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

As used herein, the term “RNA” or “RNA molecule” or “ribonucleic acid molecule” refers generally to a polymer of ribonucleotides. The term “DNA” or “DNA molecule” or deoxyribonucleic acid molecule” refers generally to a polymer of deoxyribonucleotides. DNA and RNA molecules can be synthesized naturally (e.g., by DNA replication or transcription of DNA, respectively). RNA molecules can be post-transcriptionally modified. DNA and RNA molecules can also be chemically synthesized. DNA and RNA molecules can be single-stranded (i.e., ssRNA and ssDNA, respectively) or multi-stranded (e.g., double stranded, i.e., dsRNA and dsDNA, respectively). Based on the nature of the invention, however, the term “RNA” or “RNA molecule” or “ribonucleic acid molecule” can also refer to a polymer comprising primarily (i.e., greater than 80% or, preferably greater than 90%) ribonucleotides but optionally including at least one non-ribonucleotide molecule, for example, at least one deoxyribonucleotide and/or at least one nucleotide analog.

As used herein, the term “nucleotide analog”, also referred to herein as an “altered nucleotide” or “modified nucleotide,” refers to a non-standard nucleotide, including non-naturally occurring ribonucleotides or deoxyribonucleotides. Preferred nucleotide analogs are modified at any position so as to alter certain chemical properties of the nucleotide yet retain the ability of the nucleotide analog to perform its intended function.

As used herein, the term “RNA analog” refers to a polynucleotide (e.g., a chemically synthesized polynucleotide) having at least one altered or modified nucleotide as compared to a corresponding unaltered or unmodified RNA but retaining the same or similar nature or function as the corresponding unaltered or unmodified RNA. As discussed above, the oligonucleotides may be linked with linkages which result in a lower rate of hydrolysis of the RNA analog as compared to an RNA molecule with phosphodiester linkages. Exemplary RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate (mediates) RNA interference or otherwise reduce target gene expression.

### Methods of Making Transgenic Non-Human Animals

Any of various methods can be used to introduce a transgene into a non-human animal to produce a transgenic animal. Such techniques are well-known in the art and include, but are not limited to, pronuclear microinjection, viral infection and transformation of embryonic stem cells and iPS cells. Methods for generating transgenic animals that can be used include, but are not limited to, those described in J. P. Sundberg and T. Ichiki, Eds., Genetically Engineered Mice Handbook, CRC Press; 2006; M. H. Hofker and I. van Deursen, Eds., Transgenic Mouse Methods and Protocols, Humana Press, 2002; A. L. Joyner, Gene Targeting: A Practical Approach, Oxford University Press, 2000; Manipulating the Mouse Embryo: A Laboratory Manual, 3rd edition, Cold Spring Harbor Laboratory Press; 2002, ISBN-10: 0879695919; K. Turksen (Ed.), Embryonic stem cells: methods and protocols in Methods Mol. Biol. 2002; 185, Humana Press; Current Protocols in Stem Cell Biology, ISBN: 978047015180; Meyer et al. PNAS USA, vol. 107 (34), 15022-15026.

In certain embodiments, the genetically engineered animals with site-specific knock-ins can be created using spermatogonial stem cells (SSCs), piggyBac™ mobile DNA technology using transposable elements, Xanthamonas transcription activator-like (TAL) Nucleases (XTNs) [aka TAL-effector nucleases (TALENs)], and a combination thereof.

### Methods for Customizing Animal Traits

In one embodiment, the present invention provides a method of customizing animal traits using the transgenic animal of the invention. In one embodiment, the method comprises:

a) providing a transgenic animal whose genome comprises:

an exogenous nucleic acid molecule encoding a protein of interest, wherein the nucleic acid molecule is operably linked to a promoter and is under the control of an inducible gene expression system that requires the presence of an inducing agent to activate gene expression;

wherein the expression of the exogenous nucleic acid molecule is inhibited in the absence of the inducing agent;
b) administering the inducing agent to the transgenic animal thereby inducing the expression of the exogenous nucleic acid molecule.

In certain embodiments, the exogenous nucleic acid molecule encodes a protein of interest, wherein the protein of interest is selected from pigment proteins; proteins involved in the synthesis and/or transport of pigments; luminescent (such as fluorescent) proteins; proteins involving the length and/or texture of animal skin or fur; and proteins involved in the texture, structural strength, and/or length of animal nail, claw, and/or horn.

In certain embodiments, the inducible gene expression systems useful according the present invention include, but are not limited to, site-specific recombination systems including, but not limited to, a Cre-LoxP recombination system, a FLP-FRT recombination system; a tetracycline (Tet)-controlled transcription activation system; an ecdysone inducible system; a heat shock on/off system; a lacO/IPTG system; a cumate repressor protein CymR system; a nitroreductase system; coumermycin/novobiocin-regulated system; a RheoSwitch Ligand RSL1 system; a chimeric bipartite nuclear receptor expression system; a GAL4 system; sterol or steroid or synthetic steroid inducing/repressing system; and any combinations thereof.

In certain embodiments, the inducing agents for gene expression useful according the present invention include, but are not limited to, cre recombinase, HTCre; FLP recombinase; tetracycline or its derivatives such as doxycycline; ecdysone; cumate; nitroreductase steroids; and any combinations thereof.

In one embodiment, the transgenic animal comprises an exogenous nucleic acid molecule that is under the control of a site-specific recombination system, and the expression of the exogenous nucleic acid molecule is induced after the administration (such as via topical administration) of a recombinase protein, or the administration (such as via injection) of a nucleic molecule encoding a recombinase protein, to the transgenic animal.

In one embodiment, the genome of the transgenic animal comprises an exogenous nucleic acid molecule whose expression is under the control of a Cre/LoxP recombination system, wherein the Cre/LoxP recombination system prevents the expression of the exogenous nucleic acid molecule, wherein the administration (such as via topical administration) of Cre recombinase and/or HTCre, or the administration (such as via injection) of a nucleic molecule encoding Cre recombinase and/or HTCre, to the transgenic animal, induces the expression of the exogenous nucleic acid molecule, thereby customizing the animal trait(s) of interest.

In another specific embodiment, the method of customizing animal traits comprises:

a) providing a transgenic animal whose genome comprises:

a first exogenous nucleic acid molecule encoding a protein of interest (such as pigmentation protein) operably linked to a first promoter and under the control of a loxP site, wherein the loxP site prevents the expression of the first nucleic acid molecule in the absence of Cre recombinase protein;

a second nucleic acid encoding a reverse tTA (rtTA), operably linked to a second promoter; and

a third nucleic acid molecule encoding a Cre recombinase protein, operably linked to a third promoter under the control of a TetO operator;

b) administering doxycycline to the transgenic animal, thereby inducing the expression of the exogenous nucleic acid molecule.

The inducing agent for administration to the transgenic animal can be in a form that can be combined with a carrier. The term “carrier” refers to a diluent, adjuvant, excipient, or vehicle with which the compound is administered. Such pharmaceutical carriers can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers, particularly for injectable solutions.

The inducing agent and compositions can be administered to the transgenic animal by standard routes, including oral, inhalation, or parenteral administration including intravenous, subcutaneous, topical, transdermal, intradermal, transmucosal, intraperitoneal, intramuscular, intracapsular, intraorbital, intracardiac, transtracheal, subcutaneous, subcuticular, intraarticular, subcapsular, subarachnoid, intraspinal, epidural and intrasternal injection, infusion, and electroporation, as well as co-administration as a component of any medical device or object to be inserted (temporarily or permanently) into a transgenic animal.

### EXAMPLES

Following are examples that illustrate procedures and embodiments for practicing the invention. The examples should not be construed as limiting.

### Example 1

### Customization of Skin or Fur Pigmentation in Animals

This Example provides embodiments of genetic constructs for customizing skin and fur pigmentation in animals.

Animals with Customized Pigmentation and Pattern in the Skin or Fur

C3H/HeJ murine strain with a brown (“Agouti” coloration) skin color are genetically engineered to express the following three constructs: 1) a construct comprising a keratin-14 specific promoter, a loxp cassette comprising a nucleic acid encoding a red fluorescent protein, a pigment cassette comprising a nucleic acid encoding a dominant black (ΔG23) beta defensin 103 protein, and an SV40 (with intron) polyadenylation sequence; 2) a construct comprising a nucleic acid molecule encoding a reverse tetracycline transactivator (rtTA), operably linked to a keratin-14 specific promoter that initiates the transcription of the nucleic acid encoding rtTA, and an SV40 polyadenylation sequence; and 3) a construct comprising an agouti signaling protein (ASP), operably linked to a tetracycline-sensitive promoter (TetO)7 that initiates the transcription of the nucleic acid molecule encoding ASP, and a bovine growth hormone (BGH) polyadenylation sequence.

When doxycycline is fed to the genetically-modified mice, mouse fur turns golden (this is the first demonstration of genetic modification of hair color after birth). The application of Cre or HTNCre to the shaved skin of the genetically-engineered mice, via a carrier base (e.g., protein carriers, such as lipid bilayers), induces genetically permanent black coloration of fur in the area where Cre and/or HTNCre is applied. In accordance with the present invention, mice born with brown fur can be modified to have golden fur with arbitrarily shaped black markings. For example, a black name or black logo can be created on mice fur with a gold background.

To customize skin pigmentation in animals, the animal could only express a pigmentation construct, and need not express a patterning construct (as shown in FIG. 2). The customization of pigmentations and patterns is activated directly by application of Cre and/or HTNCre.

In one embodiment heritable patterns can be created by genetically modifying the animals to express a patterning construct. FIG. 2 shows one embodiment of a patterning construct. The promoter can be the Ripply2 promoter, or from any gene specific to somite boundary specification. Alternate promoters could be the Tabby or Ticked promoters.

In one embodiment, the genetically-modified animal, whose genome comprises a pigmentation construct and a patterning construct, has constitutive vertical stripes on the dorsal dermis.

### Complex/Multicolored Patterns

FIG. 3 illustrates certain embodiments of genetic constructs for creating complex or multicolored patterns in animal skin or fur. In one embodiment, complex patterns can be created with the use of a construct comprising an inducible system, such as promoters with mechanisms of inducibility. As shown in FIG. 3, Cre is activated by the presence of doxycycline only in tissues specific for the rtTA promoter. The use of tissue-specific promoter maintains somite border in animals; for example, the transgene can only be activated during the developmental period. Inducible system can also be used to create multiple colors.

Inducible systems useful according to the present invention include, but are not limited to, tetracycline, ecdysone, and tamoxifen inducible systems; FLP-FRT recombination system; and Cre-LOX recombination system.

### Example 2

### Mice with Customized Fur Color and Pattern

This Example shows the creation of genetically-modified mice whose fur color can be permanently altered through the transdermal application of HTNCre—a recombinase that can easily cross cell membranes.

FIG. 4 shows a genetic construct for creating customized patterns and color in mouse skin or fur. The construct comprises a Keratin14 promoter, which is expressed in all skin fibroblasts, to drive the dominant black form of signaling molecule beta-Defl 03; the expression of beta-Def103 is blocked by a Loxp excisable nucleic acid encoding ring finger protein (RFP) (the RFP is used as a marker).

Agouti mice are genetically-engineered to express a transgene encoding the “dominant black” signaling molecule βDef103, and the transgene expression is activated by the application of recombinase.

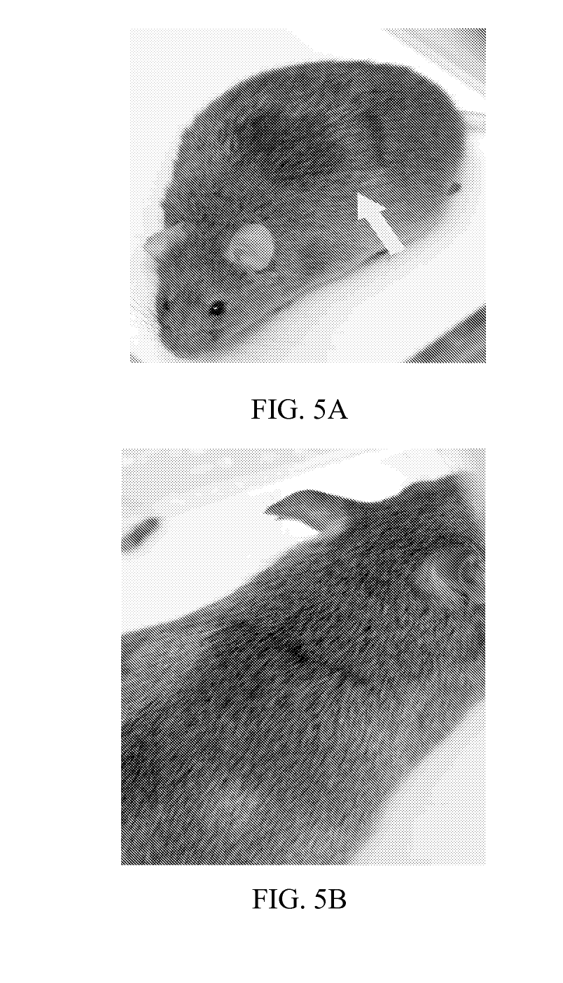

FIGS. 5A and B are photographs that show two genetically-engineered mice in which the expression of the dominant black pigment protein is activated by dermal or intradermal application of HTNCre in a carrier solution. Before the present invention, recombinase has never been applied in live animals.

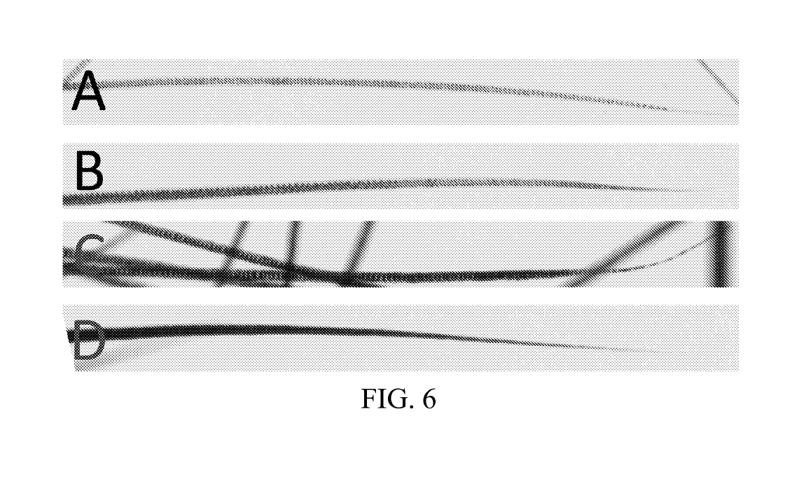

FIG. 6 shows that the application of recombinase to genetically-engineered mice can result in dose dependent change in fur color. The tip of Agouti mouse hairs are normally characterized by cells comprising yellow pheomelanin (A). As shown in FIG. 6, through increasing activation of the transgene by increasing recombinase doses, the mouse fur pigments can be shifted to a mix of pheomelanin and black eumelanin (B) to pure eumelanin (C) to so much melanin that the compartmentalized structure breaks down (D). This Example shows that transdermal application of recombinase can result in fine control of hair color in genetically-engineered mice.

### Example 3

### Customized Skin Color and Pattern for Cattle Identification

In the cattle industry, a robust method of birth processing for individual identification has become increasingly important for proof of ownership, herd management, tracking of animal movements, and perhaps, most importantly, animal disease traceability.

This Example provides transgenic cattle having customized skin color and patterns that can be used as a code (e.g., bar code) for cattle identification. The transgenic cattle can be created using the method described in Example 2. As shown in Example 2, transdermal or intradermal application of recombinase to transgenic mice whose genomic comprises a Cre-LoxP recombination system induce customizable changes in coat color after birth.

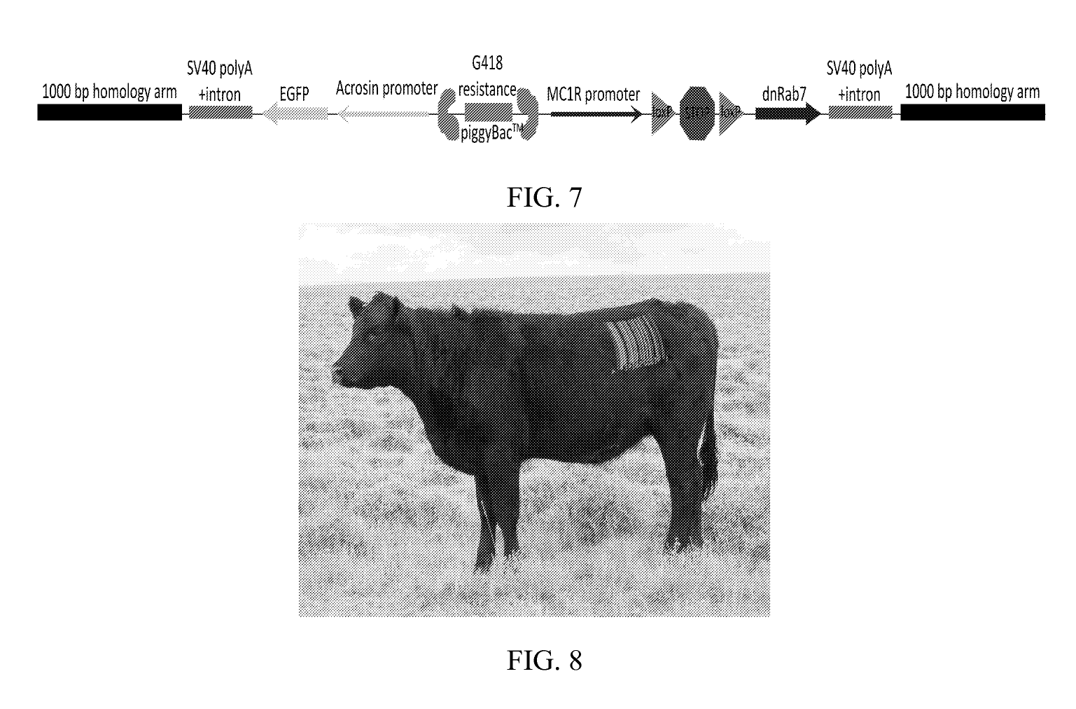

FIG. 7 shows a construct design for creating customized pattern or color identification in cattle. The construct comprises a nucleic acid molecule encoding a dominant negative Rab7, operably linked to a MC1R promoter and under the control of the loxP-STOP-loxP sequence. The expression of the Rab7 can be induced by the application of Cre recombinase. The use of TAL nucleases allows site-specific knock-ins with short homology arms. The acrosin promoter/EGFP arm on the construct allows flow sorting for genetically modified sperm, thereby ensuring 100% genetically modified offspring in the F1 generation. An important element for the construct is the MC1R (melanocortin receptor) promoter, which drives the expression of a dominant negative Rab7 gene only when activated with recombinase. The combination of various elements in the construct allows creation of cattle modified with site-specific knock-in(s) that allows creation of permanent identification information on cattle.

FIG. 8 shows a depiction of a Black Angus heifer genetically engineered to express a customizable identification pattern in the skin.

During birth processing, individual identification information can be easily created by applying Cre recombinase to cattle whose genome comprises the construct shown in FIG. 7. Also, the transgenes are only expressed in the animal skin, which can be removed during food processing; therefore, the creation of cattle identification in the animal skin should not raise regulatory issues with governmental agencies such as USDA.

In certain embodiments, site-specific knock-in animals can be created using conventional technologies, including, but not limited to, spermatogonial stem cells (SSCs), piggyBac™ mobile DNA technology using transposable elements, Xanthamonas transcription activator-like (TAL) Nucleases (XTNs) [aka TAL-effector nucleases (TALENs)], and a combination thereof

In one embodiment, Black Angus coat color is post-natally modified with a heterozygous knock-in using a melanocyte-specific dominant negative Rab7, which is required for intracellular transport of the critical melanogenesis gene Tyrp1.

### Example 4

### Customized Skin Color and Pattern for Cattle Disease Detection

Diseases are a concern for nearly every beef and dairy producer, and many common diseases can dramatically impact production without having overt clinical signs. Sudden death is often the first and only sign of clostridial diseases. Subclinical mastitis is a common and expensive problem in dairy production. Even rumors of bovine spongioform encephalopathy (BSE), which rarely has overt clinical signs, can cause severe economic damage to beef industry. In addition to infectious diseases, metabolic diseases in cattle can also cause economic damages: ⅓ of beef cattle have subclinical copper deficiency due to the presence of chelating agents in their diet. Changes in fur or skin color, in accordance with the present invention (e.g., using recombinase-activated coat-color specific markers) can be used to identify the presence of diseases in cattle.

In one embodiment, the construct for cattle disease detection is identical to the construct as shown in FIG. 7, except that the MC1R promoter is replaced with an alternate recombinase system (e.g., Flp-Frt recombination system) and/or disease-specific promoters, such as promoters responsive to NFkB, Ifnγ, or copper-deficiency targets.

In one embodiment, during birth processing, cattle are painted with the recombinase to create the desired symbol. The painted area would change color once the cattle develop inflammatory or metabolic stresses (the expression of pigment protein regulated by disease-specific promoter). The cattle disease detection application can be used in combination with the cattle identification application through the use of different recombinases and recombinase targets.

### Example 5

### Dogs with Customizable Coats

Different dog breeds have coat color as a result of different combinations of mutations in pigment genes. Pugs have an intact melanocortin receptor with no endogenous expression of the “dominant black” signaling molecule βDef103.

Dogs with customized fur color and pattern can be created using the methods described in Examples 3 and 4 for cattle, or through the process shown in Example 2 for mice. In one embodiment, genetically-engineered pugs whose genome comprises the construct as shown in FIG. 7 have fur with customizable color and pattern including tiger stripes, logos, writing, and hearts.

Dogs are normally difficult to genetically engineer due to the oddities in the egg development system in canines Currently, genetically-engineered dogs cannot be created by in vitro fertilization (IVF). Genetically-engineered dogs with site-specific knock-ins can be created using spermatogonial stem cells (SSCs), piggyBac™ mobile DNA technology using transposable elements, Xanthamonas transcription activator-like (TAL) Nucleases (XTNs) [aka TAL-effector nucleases (TALENs)], and a combination thereof.

### Example 6

### Creation of Transgenic Angus Cattle with “White” Coat Color

Heat stress is a major cause of morbidity and mortality among cattle and has adverse economic impact on the cattle industry. Dark coat color substantially worsens the animal's ability to tolerate heat stress, with black-coated cattle showing more than five times the increase in core body temperature of white cattle.

Black Angus is the most popular breed of beef cattle in the United States. Partly due to its black coat, the Black Angus breed tolerates heat stress poorly and is not suited to live in hot climates. Problems of poor heat tolerance in Black Angus has been a long-standing problem in the cattle industry.

Existing attempts to create the Angus breed with white or light coat color through breeding programs have failed, partly because the black coat color in Black Angus breed is the characteristic of a dominantly acting, constitutively active allele of MC1R, and in cattle there are no dominant white alleles downstream. A dominant downstream allele is needed to create the Angus breed with a “white” coat via outbreeding. Tyrosinase and oca mutations that occasionally occur and create albino Black Angus are not useful for creating the Angus breed with a “white” coat, because this strategy requires inbreeding.

This Example provides a method of creating Angus cattle having a “white” coat via the introduction of a heterologous, non-native dominant white allele into the cattle.

In one embodiment, a dominant white allele of Pmel17 (also called GP100 and Silv) in chicken is transferred to cattle. Pmel17 relates to melanin assembly in melanosomes. While Charolais cattle with a recessive mutation in Pmel17 have white coat color, cattle with a dominant white allele are preferred. Dominantly acting Pmel17 alleles are found in mice, dogs, and horses. Transfer of these dominant white alleles to cattle can be accomplished via a knock-in of a dominant mutation to the recessive Pmel17 allele, or through introduction of a non-native dominant white Pmel17 at a locus different from the native Pmel17 allele.

Genes useful for creating animals (such as Angus cattle) with a white coat include, but are limited to, dominant alleles involved in melanosome assembly (such as Pmel17, MLPH, and the dominant negative Rab7 allele); and alleles encoding proteins that inhibit the transport of melanin to melanosomes.

Also, transgenic animals (such as cattle) with white coat color can be created by introducing dominant mutations in genes encoding proteins involved in melanocyte migration from the neural crest during development, thereby obtaining white animals (such as cattle) that lack melanocytes.

In addition, transgenic animals (such as cattle) with white coat color can be created by introducing, into the animals, siRNA constructs (such as miRNA constructs) that inhibit the expression of nucleic acids encoding proteins for transporting melanin or proteins necessary for the migration of melanocytes.

The present invention also provides transgenic animals (such as cattle) with white coat color, wherein genetic black markings can be created by dermal or transdermal application of recombinase proteins.

Using any combinations of aforementioned ways, genes non-native to cattle can be used to create a “white” allele dominant over the constitutively active MC1R found in Black Angus, resulting in white or silver-haired offspring in an outbred cross between the engineered bull and a normal Black Angus cow.

All references, including publications, patent applications and patents, cited herein are hereby incorporated by reference to the same extent as if each reference was individually and specifically indicated to be incorporated by reference and was set forth in its entirety herein.

The terms “a” and “an” and “the” and similar referents as used in the context of describing the invention are to be construed to cover both the singular and the plural, unless otherwise indicated herein or clearly contradicted by context.

Recitation of ranges of values herein are merely intended to serve as a shorthand method of referring individually to each separate value falling within the range, unless otherwise indicated herein, and each separate value is incorporated into the specification as if it were individually recited herein. Unless otherwise stated, all exact values provided herein are representative of corresponding approximate values (e.g., all exact exemplary values provided with respect to a particular factor or measurement can be considered to also provide a corresponding approximate measurement, modified by “about,” where appropriate).

The use of any and all examples, or exemplary language (e.g., “such as”) provided herein, is intended merely to better illuminate the invention and does not pose a limitation on the scope of the invention unless otherwise indicated. No language in the specification should be construed as indicating any element is essential to the practice of the invention unless as much is explicitly stated.

The description herein of any aspect or embodiment of the invention using terms such as “comprising”, “having”, “including” or “containing” with reference to an element or elements is intended to provide support for a similar aspect or embodiment of the invention that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context).

It should be understood that the examples and embodiments described herein are for illustrative purposes only and that various modifications or changes in light thereof will be suggested to persons skilled in the art and are to be included within the spirit and purview of this application.

### REFERENCES

1. Candille S I, Kaelin C B, Cattanach B M, Yu B, Thompson D A, Nix M A, Kerns J A, Schmutz S M, Millhauser G L, and Barsh G S. A-defensin mutation causes black coat color in domestic dogs. Science 318: 1418-1423, 2007.
2. Kistner et al., Doxycycline-mediated quantitative and tissue-specific control of gene expression in transgenic mice, Proc. Natl. Acad. Sci. U.S.A. Vol. 93, pp. 10933-10938, 1996.
3. Nolden L, Edenhofer F, Haupt S, Koch P, Wunderlich F T, Siemen H, and Brustle O. Site-specific recombination in human embryonic stem cells induced by cell-permeant Cre recombinase. Nat Methods 3: 461-467, 2006.
4. Nagy, Cre recombinase: the universal reagent for genome tailoring, Genesis 26:99-109, 2000.

1. A cell of a non-human transgenic animal with coat or fur having a color of interest, wherein the transgenic animal comprises:
an exogenous nucleic acid molecule, operably linked to a promoter, wherein the exogenous nucleic acid encodes a protein selected from proteins involved in melanosome assembly, proteins involved in the synthesis of melanin, proteins involved in the transport of melanin, and proteins involved in melanocyte development and/or migration; and/or
an exogenous inhibitory RNA coding sequence of interest, operably linked to a promoter, wherein the exogenous inhibitory RNA coding sequence encodes an inhibitory RNA that interferes with the expression of a wild-type nucleic acid molecule of the animal, and wherein the wild-type nucleic acid molecule encodes a protein selected from proteins involved in melanosome assembly, proteins involved in the synthesis of melanin, proteins involved in the transport of melanin, and proteins involved in melanocyte development and/or migration.

2. The cell, according to claim 1, comprising exogenous nucleic acid molecule, operably linked to a promoter, wherein the exogenous nucleic acid encodes a protein selected from proteins involved in melanosome assembly, proteins involved in the synthesis of melanin, proteins involved in the transport of melanin, and proteins involved in melanocyte development and/or migration.

3. The cell, according to claim 1, comprising an exogenous inhibitory RNA coding sequence of interest, operably linked to a promoter, wherein the exogenous inhibitory RNA coding sequence encodes an inhibitory RNA that interferes with the expression of a wild-type nucleic acid molecule of the animal, and wherein the wild-type nucleic acid molecule encodes a protein selected from proteins involved in melanosome assembly, proteins involved in the synthesis of melanin, proteins involved in the transport of melanin, and proteins involved in melanocyte development and/or migration.

4. A non-human transgenic animal comprising a cell according to claim 1.

5. The non-human transgenic animal, according to claim 4, which is a dog, cat, mouse, rat, guinea pig, hamster, horse, bovine, pig, sheep, goat, duck, goose, chicken, primate, fish, frog, salamander, snake, lizard, fox, weasel, rabbit, mink, beaver, ermine, otter, sable, seal, coyote, chinchilla, deer, muskrat, or possum.

6. The non-human transgenic animal, according to claim 4, which is a bovine animal.

7. The non-human transgenic animal, according to claim 6, which is a bovine animal of the Black Angus breed.

8. A transgenic bovine animal having a white or near-white coat or fur color, according to claim 6, comprising an exogenous nucleic acid molecule that encodes a protein selected from proteins that inhibit melanosome assembly, proteins that inhibit the synthesis of melanin, and proteins that inhibit the transport of melanin, proteins that inhibit melanocyte development and/or migration.

9. The transgenic bovine animal, according to claim 8, wherein the exogenous nucleic acid molecule is a dominant white allele.

10. The transgenic bovine animal, according to claim 9, wherein the dominant white allele is a dominant white Pmel17 allele.

11. The transgenic bovine animal, according to claim 10, wherein the dominant white Pmel17 allele is from Gallus gallus.

12. The non-human transgenic animal, according to claim 4, wherein the exogenous inhibitory RNA interferes with the expression of a wild-type nucleic acid molecule encoding a protein selected from melanocortin receptor (MC1R), melanocyte stimulating hormones (MSH), β-defensin 103, agouti signaling protein (ASP), tyrosinase (TYR), melanocyte-specific transporter protein, Ras-related protein Rab-7, rab protein geranylgeranyltransferase component A2, probable E3 ubiquitin-protein ligase (HERC2), bifunctional enzyme CarRP-like, lycopene cyclase/phytoene synthase-like, and phytoene dehydrogenase-like.

13. The non-human transgenic animal, according to claim 12, which is a bovine animal.

14. The non-human transgenic animal, according to claim 13, which is a bovine animal of the Black Angus breed.

15. The non-human transgenic animal, according to claim 4, wherein the promoter is selected from universal promoters, constitutive promoters, tissue-specific promoters, and inducible promoters.

16. The non-human transgenic animal, according to claim 4, wherein the promoter is selected from cytomegalovirus (CMV) promoter, CMV-chicken beta actin promoter, ubiquitin promoter, JeT promoter, SV40 promoter, beta globin promoter, elongation Factor 1 alpha (EF1-alpha) promoter, RSV promoter, Ripply2 promoter, Ticked promoter, Tabby promoter, Mo-MLV-LTR promoter, Rosa26 promoter, keratinocyte specific promoters, melanocyte specific promoters, matrix-cell specific promoters, and dermal papilla-specific promoters.

17. The non-human transgenic animal, according to claim 4, wherein the expression of the exogenous nucleic acid molecule or the expression of the exogenous inhibitory RNA coding sequence is under the control of an inducible system selected from a Cre-LoxP recombination system, a FLP-FRT recombination system, a tetracycline (Tet)-controlled transcription activation system, an ecdysone inducible system, a heat shock on/off system, a lacO/IPTG system, a cumate repressor protein CymR system, a nitroreductase system, coumermycin/novobiocin-regulated system, a RheoSwitch Ligand RSL1 system, a chimeric bipartite nuclear receptor expression system, a GAL4 system, sterol or steroid or synthetic steroid inducing/repressing system, and any combination thereof.

18. The non-human transgenic animal, according to claim 17, wherein the inducible gene expression system comprises a Cre-LoxP recombination system.


SEQUENCE LISTING (22 sequences)

SEQ ID NO: 1 - protein, 608 aa, Acyrthosiphon pisum
MLTYIDVHFIYTLPVVAVLALITWPFISRLELFKIGFVCTMAFVYTTPWDNYIIFHNAWMYKPKNILAVIGYVPVEEYMFFVIQTLMTSLWALVFTRWSPACFNFNFNKTSYTLIRWIPILALVMTTIQGYNIAVPGKNTFYLGCIMWWSCPVIMFLWYGAGNYFVKKSTSSAIAVIVPTLYLCWVDRIALKDDVWHINEKTSLNIFVVDDLPFEECLFFLITNVIIVLGGMAFDKSYGLADTYTFEFPLRYSSSWKYYSQQMQQFVRAECDMSPSPVNDIRQCLNVLKRASKSFNVASLVFPAGVRLHLIILYAFCRVTDDMIDSEPKVGVKKQKLKLIETFIDELFADRSADYDVKTSMTPRKPEVKWEQYRLDLTDEELSCFRAISRISFYLPRKPFYELLDGYRWDVDGKTVQNETDLLLYSSYVAGSVGTLCVYVMVYKSGTQIDDDKRHDFVIGKAQQMGQVLQIVNISRDIVTDSETLGRCYVPAEYMDNAAAVVNTLCSDRDPWTLGSEKLKSYATRMIRLANRYQLESLEGIRYLPYEVRGPVLVATDIYRGVACAVEASPTYPRRASLGKWDKILVSINSLYFKSLKYFFQADRCKHC

SEQ ID NO: 2 - protein, 588 aa, Acyrthosiphon pisum
MLTYIGAHLSYTLPVIGVLTLITRPFINRLEIFKIGFISGLAVIYTIPWYSYFVYNHGARTYSPGAVLAVVGNVPVEEYMFVVMQTVLTSLWALMFVQWSTPCLNFNYDKRSYQLIRWIPISLLTVVTAVGYAMAVRGQETFYLGSILCWASPAIAIMWYGAGNFFAKKIIPSSIAIAGPTLYMCWIDRMAVSDDNNDSGKLAPEDALFVFVTNLMVVLAGSSYDKAYGMIVTYSLDFPHQFSVSCRFVRQMLRAFMTSEYATPSAVTQDIKTSIKVLSTSNAFGTSNYLFHAGIRLDLIILYAFCRVTDEMFDSKSDEKKKKLKLELSKQFISEVFADRKSDYDVKKTPQEVKIDWKKYESEFTDVELSSYRAVSRIAFFLPRKPFEELFAGYQWDLEFTLVRNEKDLMLYTTYVAGSIGAMCLYVIMYRYGNDMNDLVDKADYLTKHAYKIGQGLQLVNIARDLVSDSESLGRCYFPAEYMDDEKEDLRILCKEKNPRSLGNKKLKKYSSKMIQLANKQQFESMGAIKYLPQDLIGSVLASTEMYRGLIKAIQSCPTYPTRASLPKLSKLLIVLNTLYIKSIQYIF

SEQ ID NO: 3 - protein, 589 aa, Acyrthosiphon pisum
MLTYIDFHLKYTLTVIGVLSLIARPFINRSEVFKIAFISAIAFVYTTPWDNYVIYSDAWNYPLDRVLATIGYVPIEEYMFFIIQTVLTSLWALLCVRWSTPCLNFNYDKRSYQLIRWVPITILAIVTIVGYKLVIPGQGTFYLGCILCWVSPVIIFLWYGAGNFFVKKIIPSTFAIVVPSLYLCWIDQLALKENVWHINEKTSLNIFIVDDLPIEEAFFFFVVNLIIVLVGACFDKASGVIETYTSEYPLRFSISWKYVCQLFSAFATSEYNMPHIVTEDIKESIEIITVASKSFTTASFLFPAGIRLDLIILYSFCRVTDDMIDDELDVEKKKRKFELTERFIKELFHDRKSDYDVQTKPQELKIDWTKYESELTDREMSCFRALSRIAFYLPRKPFDELLAGYKWDIEGRLIRNEDDLLLYSTYVAGSVGALCVYVMMYRCDNDKYDLVENYDYVIEKAYQMGRALQLVNIARDIVTDSETLGRCYVPTEYMDDEEEEIRILCHEKQPRSLGDKKLKKYSTRLIHLANKQQLESLDAIRCLPHVTRGSLVKRNRGRDWNFDDLYGSGSYSVPSKAKILVSIPRKTNI

SEQ ID NO: 4 - protein, 528 aa, Acyrthosiphon pisum
MAIKIIIIGSGVGGTAAAARLSKKGFQVEVYEKNSYNGGRCSIIRHNGHRFDQGPSLYLMPKIFEETFKDLGEDIKDHIEILQCKINYYINFHDGQQFQHSCNLSKLQRSLENFEGEGEETLLRFFDFLKETHVHYRKSIELAMRTDFQNWYDFFNIKHIPTLLNLHLHSSVYTRACKYFKSDYMRKAFTFQTMYMGMSPYDGLAPYNLLQYTEIAEGIWYPKGGFHSVLESLEKIAVKHGAKFNYNSDVQEIITDENGVAKGIKLQNGNVINSDIVICNADAVYAYNKLLPKTSYAEKLGKKKLTSSSISFYWSINQVIPQMSVHNIFLSEQYKPSFDQIFEDHSLPDEPSFYVNVPSHIDPTAAPEGKDTFVILVPVGHISDRTDIDFDDLVKRAREHVINSIEKRLKISNFRSMIEHEMVNDPRTWQSEFNLWKGSVLGLSHSFFQVAYFRPSLKCKIFENLYFVGASVQPGTGVPVVLCGAKLLEKQLCARFLEGKVEMNTWSKYVSFLIGLLVLLIFWFFFRF

SEQ ID NO: 5 - protein, 528 aa, Acyrthosiphon pisum
MVVKIIIIGAGVGGTAAAARLSKRGFQVEVFEKNAYNGGRCSLIQHKGHRFDQGPSLYLMPKIFEETFEDLGEDIKNHIDLLKCPSNYSVHFHDGETFELTTDISKLSRSLEKYEGSGESTLINFLSYLKETHVHYQRSVKVALKTDFQHWYDFFNPKHIPDVIQLHLLDTVYNRVCKYFKSDYMRKAFSFQTMYLGMSPYDGLAPYSLLQYTEIAEGIWYPKGGFNKVLQSLEQIAVQYGAKFNYKTNVQEIIVDDKGVAKGIKMVNGDVVNSDIVICNADLVYAYNKLLPKTSYANKLGKKELTSSSISFYWSMKTIVPQLKVHNIFLAEKYKESFDQIFKDHTLPDEPSFYVNVPSRIDPSAAPEGKDTIVVLVPVGHISNVPNIDFDKLVERAREQVIDTIEKRLKISNFRSMIDHEIVNDPRTWQNEFNLWKGSILGLSHSLFQVLWFRPSLKCKIFENLYFVGASAQPGTGVPIVLCGAKMLEKQLCDRFLDSKVEISIWSKCVSFLIGLLALLIFWFFFRF

SEQ ID NO: 6 - protein, 526 aa, Acyrthosiphon pisum
MVVKIIIIGAGVGGTAAAARLSKKGFQVEIYEKNAYNGGRCSLIYQNGHRFDQGPSLYLMPKIFEEIFEDLGEDIKNHIDLLKCPSNYSVHFHDGETFELTTDISKLSRSLEKYEGYGESTLINFLRYLKETHVHYQRSVKVALKTDFQHWYDFFNPKFLPDVIQLHLLDTVYNRVCKYFKSDYMRKAFSFQTMYLGMSPYDGLAAYSLLQYTEIAEGIWYPKGGFHKVLESLENIAVQHGAKFNYNADVQEIIVDDKGVAKGIKMVNGDVVNSDIVICNADLVYAYNKLLPKTSYADKLGKKELTSSSISFYWSMKTIVSQLKVHNIFLAEKYKESFDQIFKDHTLPDEPSFYVNVPSRIDPTAAPEGKDTIVVLVPVGHISNVPNIDFDQHVKTAREHVIDTIEKRLKISNFRSMIDHEIVNDPRTWQNNFNLWKGSILGLSHSLFQVLWFRPSMKCKIFENLYFVGASVQPGTGVPIVLCGTKLLEKQLCDRFLDSKVTKSSWSMCVSFLIGIIVLLIFCTLF

SEQ ID NO: 7 - protein, 510 aa, Acyrthosiphon pisum
MVVKIIIIGSGVGGTAVAARLSKKGFQVEIYEKNSYNGGRCSLIYQNGHRFDQGPSLYLMPKIFEETFEDLGEDIKNHIELLKCPTNYSVHFHDGETFELTTDISKLSRSLEKYEGSGESTLINFLNYLKLTHLYYRKSVNVIQLHLLDTVYNKVSKYFKSDYMRKAFSFQTMYLGMSPYDGLALYSLLQYTEIAEGIWYPKGGYHKVLEILEKIAVQHGAKFNYNADVQEIIIDDKGVAKGIKLVNGDVVNSDIVICNADLTYAYNKLLPKTSYAEKLDKKEHTSSSISFYWSMNTIVSQLNVHNIFLAEKYKESFDQIFKDHTLPDDPSFYVNVPSRIDPTAAPEGKDSIVVLVPVGHLSNEPNIDFDKLVNKAREQVIDTIEKRLKISNFRSMIDHEKVNDPRTWRNEFNLWKGSILGLSHTFLQVVWFRPSLKCNIFKNLYFVGASAHPGTGVPVVLCGAKLLENQLCDRFLKSKAKLSLWSKCVSFLISLLTLLFLWISLFFNKT

SEQ ID NO: 8 - protein, 315 aa, Mus musculus
MSTQEPQKSLLGSLNSNATSHLGLATNQSEPWCLYVSIPDGLFLSLGLVSLVENVLVVIAITKNRNLHSPMYYFICCLALSDLMVSVSIVLETTIILLLEAGILVARVALVQQLDNLIDVLICGSMVSSLCFLGIIAIDRYISIFYALRYHSIVTLPRARRAVVGIWMVSIVSSTLFITYYKHTAVLLCLVTFFLAMLALMAILYAHMFTRACQHAQGIAQLHKRRRSIRQGFCLKGAATLTILLGIFFLCWGPFFLHLLLIVLCPQHPTCSCIFKNFNLFLLLIVLSSTVDPLIYAFRSQELRMTLKEVLLCSW

SEQ ID NO: 9 - protein, 13 aa, Artificial sequence
  Alpha melanocyte stimulating hormone
SYSMEHFRWGKPV

SEQ ID NO: 10 - protein, 22 aa, Homo sapiens
AEKKDEGPYRMEHFRWGSPPKD

SEQ ID NO: 11 - protein, 18 aa, Sus scrofa
DEGPYKMEHFRWGSPPKD

SEQ ID NO: 12 - protein, 12 aa, Artificial sequence
  gamma melanocyte stimulating hormone
YVMGHFRWDRFG

SEQ ID NO: 13 - protein, 67 aa, Mus musculus
MKLLLLTLAALLLVSQLTPGDAQKCWNLHGKCRHRCSRKESVYVYCTNGKMCCVKPKYQPKPKPWMF

SEQ ID NO: 14 - protein, 131 aa, Mus musculus
MDVTRLLLATLVGFLCFFTVHSHLALEETLGDDRSLRSNSSMNSLDFSSVSIVALNKKSKKISRKEAEKRKRSSKKKASMKKVARPPPPSPCVATRDSCKPPAPACCDPCASCQCRFFGSACTCRVLNPNC

SEQ ID NO: 15 - protein, 533 aa, Mus musculus
MFLAVLYCLLWSFQISDGHFPRACASSKNLLAKECCPPWMGDGSPCGQLSGRGSCQDILLSSAPSGPQFPFKGVDDRESWPSVFYNRTCQCSGNFMGFNCGNCKFGFGGPNCTEKRVLIRRNIFDLSVSEKNKFFSYLTLAKHTISSVYVIPTGTYGQMNNGSTPMFNDINIYDLFVWMHYYVSRDTLLGGSEIWRDIDFAHEAPGFLPWHRLFLLLWEQEIRELTGDENFTVPYWDWRDAENCDICTDEYLGGRHPENPNLLSPASFFSSWQIICSRSEEYNSHQVLCDGTPEGPLLRNPGNHDKAKTPRLPSSADVEFCLSLTQYESGSMDRTANFSFRNTLEGFASPLTGIADPSQSSMHNALHIFMNGTMSQVQGSANDPIFLLHHAFVDSIFEQWLRRHRPLLEVYPEANAPIGHNRDSYMVPFIPLYRNGDFFITSKDLGYDYSYLQESDPGFYRNYIEPYLEQASRIWPWLLGAALVGAVIAAALSGLSSRLCLQKKKKKKQPQEERQPLLMDKDDYHSLLYQSHL

SEQ ID NO: 16 - protein, 833 aa, Mus musculus
MRLENKDIRLASAVLEVELHQTSALSVPTCPDPGRLLTVKPATSNYKLGQADPCIPYAGEAAGKSVCVPEHTEFGSFLVKGSSSLKDLSFKEDTPLLWNSSQKKRSQLMPVHHPEFIATEGSWENGLTAWEQKCMLGKEVADLSALASSEKRDLAGSVHLRAQVSKLGCCVRWIKITGLFVFVVLCSILFSLYPDQGKFWQLLAVSPLENYSVNLSGHADSMILQLDLAGALMAGGPSGSGKEEHVVVVVTQTDAAGNRRRRPQQLTYNWTVLLNPRSEHVVVSRTFEIVSREAVSISIQASLQQTRLVPLLLAHQFLGASVEAQVASAVAILAGVYTLIIFEIVHRTLAAMLGALAALAALAVVGDRPSLTHVVEWIDFETLALLFGMMILVAVFSETGFFDYCAVKAYQLSRGRVWAMIFMLCLMAAILSAFLDNVTTMLLFTPVTIRLCEVLNLDPRQVLIAEVIFTNIGGAATAIGDPPNVIIVSNQELRKMGLDFAGFTAHMFLGICLVLLVSFPLLRLLYWNKKLYNKEPSEIVELKHEIHVWRLTAQRISPASREETAVRGLLLEKVLALEHLLAQRLHTFHRQISQEDKNWETNIQELQRKHRISDRSLLVKCLTVLGFVISMFFLNSFVPGIHLDLGWIAILGAIWLLILADIHDFEIILHRVEWATLLFFAALFVLMEALTHLHLVEYVGEQTALLIKMVPEDQRFAAAIVLIVWVSALASSLIDNIPFTATMIPVLLNLSQDPEISLPALPLMYALALGACLGGNGTLIGASTNVVCAGIAEKHGYGFSFMEFFRLGFPVMLMSCTIGMCYLLIAHIVVGWN

SEQ ID NO: 17 - protein, 621 aa, Mus musculus
MAEKLPTEFDVVIIGTGLPESILAAACSRSGQRVLHVDSRSYYGGNWASFSFTGLQSWLKDYQQNHDSEEGVTATWQDLIHETEEAISLRKKDETIQHTEVFCYASQDVEDSVQDTETLQRSSPLEASATPADSLDSASLPKERQSAYSTSYEVPSRHTEESDRELSLPSANVEDSLEKEKYCGDKTDMHTVSGEDKGEHKLVVQDSIEQPKRNRITYSQMVKESRRFNIDLVSKPLYSQGSLIDLLIKSNVSRYAEFKNVTRILAFWEGKVEQVPCSRADVFNSKELSMVEKRMLMKFLTFCLDYEQHSDEYQDFKQCSFSDYLKTKKLTPNLQHFILHSIAMTSESSCTTLDGLQATKTFLQCLGRFGNTPFIFPLYGHGEIPQCFCRMCAVFGGVYCLRHKVQCLVVDKDSGRCKGIIDAFGQRISANYFIVEDSYLPKETCSNVQYKQISRAVLITDQSILKTDSDQQISILVVPPLEPGTTSVRVMELCSSTMTCMKDSYLVHLTCSSSKTAREDLEPVVKQLFIPEAEAEAGKDELRKPRLLWALYFNMRDSSGVSRSSYCGLPSNVYICSGPDWGLGSEHAVKQAETLFQEIFPSEEFCPPPPNPEDIIFEAEG

SEQ ID NO: 18 - protein, 207 aa, Mus musculus
MTSRKKVLLKVIILGDSGVGKTSLMNQYVNKKFSNQYKATIGADFLTKEVMVDDRLVTMQIWDTAGQERFQSLGVAFYRGADCCVLVFDVTAPNTFKTLDSWRDEFLIQASPRDPENFPFVVLGNKIDLENRQVATKRAQAWCYSKNNIPYFETSAKEAINVEQAFQTIARNALKQETEVELYNEFPEPIKLDKNDRAKASAESCSC

SEQ ID NO: 19 - protein, 1057 aa, Mus musculus
MLCWGNASYGQLGLGGIDEEIVLEPRRSDFFVNKKVRDVGCGLRHTVFVLDDGTVYTCGCNDLGQLGHEKSRKKPEQVVALDAQNIVAVACGEAHTLALNDKGQVYAWGLDSDGQLGLQGSEECIRVPRNIKSLSDIQIVQVACGYYHSLALSKASEVFCWGQNKYGQLGLGIDCQKQTSPQLIKSLLGIPFMQVAAGGAHSFVLTLSGAIFGWGRNKFGQLGLNDENDRYVPNLLKSLRSQKIVYICCGEDHTAALTKEGGVFTFGAGGYGQLGHNSTSHEINPRKVFELMGSIVTQVACGRQHTSAFVPSSGRIYSFGLGGNGQLGTGSTSNRKSPFTVKGNWFSYNGQCPQDIGSEDYFCVKRIFSGGDQSFSHYSSPQNCGPPDDFRCSDPSKQIWTVNEALIQKWLSYPSGRFPVEIANEIDGTFSSSGCLNGSFLAISNDDHYRTGTRFSGVDMNAARLLFHKLIQPDHPQISQQVAASLEKNLIPKLTSSLPDVEALRFYLTLPECPLMSDCNNFTTIAIPFGTALVNLEKAPLKVLENWWSVLEPPLFLKIVELFKEVVVHLLKLYKIGIPPSERRIFNSFLHTALKVLEILHRVNEKTGQLIQYDKFYIHEVQELIDIRNDYINWVQQQAYGVDVSHGVTELADIPVTICTYPFVFDAQAKTTLLQTDAVLQMQMAIDQAHRQNVSSLFLPVIESVNPCLILVVRRENIVGDAMEVLRKTKNIDYKKPLKVIFVGEDAVDAGGVRKEFFLLIMRELLDPKYGMFRYYEDSRLIWFSDKTFEDSDLFHLIGVICGLAIYNFTIVDLHFPLALYKKLLKRKPSLDDLKELMPAVGRSMQQLLDYPEDDIEETFCLNFTITVENFGATEVKELVLNGADTAVNRQNRQEFVDAYVDYIFNKSVASLFDAFHAGFHKVCGGKVLLLFQPNELQAMVIGNTNYDWKELEKNTEYKGEYWADHPTIKIFWEVFHELPLEKKKQFLLFLTGSDRIPILGMKSLKLVIQSTGGGESYLPVSHTCFNLLDLPKYTEKETLRCKLIQAIDHNEGFSLI

SEQ ID NO: 20 - protein, 783 aa, Gallus gallus
MRLHGAIVLLAALLALVTAQQRGGGRSRGGVKGSVWGGRPAPFRSWDTARYRPWQEGTARQNDCWRGGDVTFDISNDAPTLVGARATFSIALRFPSTQTVLPDGRVVWSQNCTVNGTRMLQGDPVYPEQLAEGSDGVFPDGQPFPRSAWGKRGRFVYVWWTWGRYWQVVDGATSQLTVGTDGVALGSYTMEVVVYHYRGRQRFIPIGHASTQFSITDQVPIAVDVTQLEVAAGDGGSFVRNRPVAFNVRLHDPSHYLRDADISYSWDFGDQSGTLISRSPTVTHTYLQAGSFAARLVLQAAIPLSSCGTSAPPVVDPTTGPVPSLGPTATQPVGPTGSGTATAPSNLTGSGTAAAPGTTAAPRASGAPAEPTGVSVAVLSDSAATEPLPDPVLSTAVANAAAGTDPTADPLPPTSVSSGGDAPGTVAPTAVEGSVAAGVGTAEDVAAATPGATAADVAVDTAGATDGDAVGPTAAATAESIADPTAGATDGDAVGPTAAATAESIADPTAGATDGDAVGPTAAATAESIADPIVGATDGDAVGPTAAATAESIADPTAGATAVSSGSATAGATAEPLLLVKRQAPEAEPTGCVLYRYGTFSTELNIVQGIESVAIVQVVPAAPEGSGNSVELTVTCEGSLPEEVCTVVADAECRTAQMQTCSAVAPAPGCQLVLRQDFNQSGLYCLNVSLANGNGLAVASTHVAVGGASPAASGTTLTVGLLLIAAAYTYRCVKYSPLLPTAPTAPRPHSWLPPGATLRLLLRQAFGGAPSGESSPLLRANAV

SEQ ID NO: 21 - protein, 760 aa, Gallus gallus
MRLHGAIVLLAALLALVTAQQRGGGRSRGGVKGPAWGGRPAPFRSWDTARYRPWQEGTARQNDCWRGGDVTFDISNDAPTLVGARATFSIALRFPGTQTVLPDGRVVWSQNCTVNGTRMLQGDPVYPEQLAEGSDGVFPDGQPFPRSAWGKRGRFVYVWWTWGRYWQVVDGATSQLTVGTDGVALGSYTMEVVVYHYRGRQRFIPIGHASTQFSITDQVPIAVDVTQLEVAAGDGGSFVRNRPVAFNVRLHDPSHYLRDADISYSWDFGDQSGTLISRSPTVTHTYLQAGSFAARLVLQAAIPLSSCGTSAPPVVDPTTGPVPSLGPTATQPVGPTGSGTATAPSNLTGSGTAAAPGTTAAPRASGAPAEPTGVSVAVLSDSAATEPLPDPVLSTAVADAAAGTDPTADPLPPTSVSSGGDAPGTVAPTAVEGSVAAGVGTAEDVAAATPGATAADVAVDTAGATDGDAVGPTAAATAESIADPTAGATDGDAVGATAESIADPTAGATDGDAVGPTAAATAESIADPTAGATAVSSGSATAGATAEPLLLVKRQAPEAEPTGCVLYRYGTFSTELNIVQGIESVAIVQVVPAAPEGSGNSVELTVTCEGSLPEEVCTVVADAECRTAQMQTCSAVAPAPGCQLVLRQDFNQSGLYCLNVSLANGNGLAVASTHVAVGGASPAASGTTLTVGLLLIAAALGTAAYTYRRVKYSPLLPTAPTAPRPHSWLPPGATLRLLLRQAFGGAPSGESSPLLRANAV

SEQ ID NO: 22 - protein, 759 aa, Gallus gallus
MRLHGAIVLLAALLALVTAQQRGGGRSRGGVKGSAWGGRPAPFRSWDTARYRPWQEGTARQNDCWRGGDVTFDISNDAPTLVGARATFSIALRFPGTQTVLPDGRVVWSQNCTVNGTRMLQGDPVYPEQLAEGSDGVFPDGQPFPRSAWGKRGRFVYVWWTWGRYWQVVDGATSQLTVGTDGVALGSYTMEVVVYHYRGRQRFIPIGHASTQFSITDQVPIAVDVTQLEVAAGDGGSFVRNRPVAFNVRLHDPSHYLRDADISYSWDFGDQSGTLISRSHTYLQAGSFAARLVLQAAIPLSSCGTSAPPVVDPTTGPVPSLGPTATQPVGPTGSGTATAPSNLTGSGTAAAPGTTAAPRASGAPAEPTGVSVAVLSDSAATEPLPDPVLSTAVADAAAGTDPTADPLPPTSVSSGGDAPGTVAPTAVEGSVAAGVGTAEDVAAATPGATAADVAVDTAGATDGDAVGPTAAATAESIADPTAGATDGDAVGATAESIADPTAGATDGDAVGPTAAATAESIADPTAGATAVSSGSATAGATAEPLLLVKRQAPEAEPTGCVLYRYGTFSTELNIVQGIESVAIVQVVPAAPEGSGNSVELTVTCEGSLPEEVCTVVADAECRTAQMQTCSAVAPAPGCQLVLRQDFNQSGLYCLNVSLANGNGLAVASTHVAVGGASPAASGTTLTVGLLWAPLIAAALGTAAYTYRRVKYSPLLPTAPTAPRPHSWLPPGATLRLLLRQAFGGAPSGESSPLLRANAV